(19) United States
(12) Patent Application Publication
(10) Pub. No.: US 2024/0271129 A1
(43) Pub. Date: Aug. 15, 2024

(54) RNA-GUIDED CAS NUCLEASES AND USES THEREOF IN DIAGNOSTICS AND THERAPY

(71) Applicants: HELMHOLTZ-ZENTRUM FÜR INFEKTIONSFORSCHUNG GMBH (HZI), BRAUNSCHWEIG (DE); UTAH STATE UNIVERSITY, LOGAN, UT (US)

(72) Inventors: Chase BEISEL, WUERZBURG (DE); RYAN JACKSON, LOGAN, UT (US); OLEG DMYTRENKO, WUERZBURG (DE)

(21) Appl. No.: 18/564,684

(22) PCT Filed: Jun. 1, 2022
(86) PCT No.: PCT/EP2022/064930

§ 371 Date: Nov. 28, 2023

Related U.S. Application Data
(63) Continuation of application No. 17/335,818, filed on Jun. 1, 2021.

(51) Int. Cl. C12N 15/11; A61K 31/7088; C12N 9/22

(52) U.S. Cl. CPC C12N 15/111; C12N 9/22; A61K 31/7088; C12Q 2521/301; C12Q 2560/00

(57) ABSTRACT

The present invention relates to methods for RNA-directed cleaving of a nucleic acid molecule selected from dsDNA, ssDNA, and RNA based on a complex comprising a CasΩ nuclease and at least one pre-selected guide RNA designed for binding to at least one target RNA. Further provided is the complex of the present invention bound to a target RNA molecule, as well as respective systems for cleaving of a nucleic acid molecule, and diagnostic and therapeutic uses thereof.

The present invention relates to methods for RNA-directed cleaving of a nucleic acid molecule selected from dsDNA, ssDNA, and RNA based on a complex comprising a CasΩ nuclease and at least one pre-selected guide RNA designed for binding to at least one target RNA. Further provided is the complex of the present invention bound to a target RNA molecule, as well as respective systems for cleaving of a nucleic acid molecule, and diagnostic and therapeutic uses thereof.

### BACKGROUND OF THE INVENTION

Almost all archaea and about half of bacteria possess clustered regularly interspaced short palindromic repeat (CRISPR)-CRISPR-associated genes (Cas) adaptive immune systems, which protect prokaryotes against viruses and other foreign invaders with nucleic-acid genomes. The CRISPR-Cas system is functionally divided into classes 1 and 2 according to the composition of the effector complexes. Class 2 consists of a single-effector nuclease, and routine practice of genome editing has been achieved by the exploitation of Class 2 CRISPR-Cas systems, which include the type II, V, and VI CRISPR-Cas systems. Types II and V are principally used for targeting DNA, while type VI is employed only for targeting RNA (see, for example, Koonin E V and Makarova K S Origins and evolution of CRISPR-Cas systems Philos Trans R Soc Lond B Biol Sci. 2019 May 13; 374(1772):20180087).

Types II and V Cas effector nucleases commonly rely on protospacer-adjacent motifs (PAMs) as the first step in target DNA recognition, and the effector nucleases directly bind the PAM sequence through protein-DNA interactions and subsequently unzip the downstream DNA sequence. The effector proteins then interrogate the extent of base pairing between one strand of the DNA target and the guide portion of the CRISPR RNA (crRNA). Sufficient complementarity between the two drives target cleavage. PAM sequences are known to vary considerably not only between systems but also between otherwise similar nucleases, and it was shown that Cas proteins can be engineered to alter PAM recognition (Collias, D., Beisel, C. L. CRISPR technologies and the search for the PAM-free nuclease. Nat Commun 12, 555 (2021). https://doi.org/10.1038/s41467-020-20633-y). In addition to targeting DNA, some Type II and V single-effector nucleases such as the C. jejuni Cas9, the N. meningitidis Cas9, the S. aureus Cas9, Cas12f1 from an uncultured archaeon, and Cas12g have also been shown to target ssDNA and/or RNA (RNA-dependent RNA targeting by CRISPR-Cas9. Elife. 2018; 7:e32724; DNase H Activity of Neisseria meningitidis Cas9. Mol Cell. 2015; 60(2):242-255; Programmed DNA destruction by miniature CRISPR-Cas14 enzymes; Functionally diverse type V CRISPR-Cas systems. Science. 2019; 363:88-91). In these cases, no PAM was required. Some nucleases such as the S. pyogenes Cas9 (SpyCas9) were not able to immediately target ssDNA or RNA, although providing an oligonucleotide to generate a double-stranded PAM region allowed SpyCas9 to bind the single-stranded target and cleave it (Programmable RNA recognition and cleavage by CRISPR/Cas9. Nature. 2014; 516(7530):263-266).

Cas13 proteins, such as Cas13a of Leptotrichia shahii (formerly C2c2), bind and cleave RNA instead of DNA and bind to a Protospacer Flanking Site (PFS) instead of a PAM. In vivo studies have shown that target RNAs with extended complementarity with repeat sequences flanking the target element (tag:anti-tag pairing) can dramatically reduce RNA cleavage by the type VI-A Cas13a system, defining the molecular principles underlying Cas13a's capacity to target and discriminate between self and non-self RNA targets (Wang B, Zhang T, Yin J, Yu Y, Xu W, Ding J, Patel D J, Yang H. Structural basis for self-cleavage prevention by tag:anti-tag pairing complementarity in type VI Cas13 CRISPR systems. Mol Cell. 2021 Mar. 4; 81(5):1100-1115.e5. doi: 10.1016/j.molcel.2020.12.033. Epub 2021 Jan. 19. PMID: 33472057).

In the context of the present invention, removing the flanking sequence from the target RNA abolishes cleavage activity, and the flanking sequence is thus activating for CasQ, and also seems to require a specific sequence (versus lack of complementarity with the guide RNA tag). The role of this flanking sequence is most closely related to an rPAM (for RNA PAM), which have been reported for type III CRISPR-Cas systems that encode multi-subunit effectors (see Elmore J R, et al. Bipartite recognition of target RNAs activates DNA cleavage by the Type III-B CRISPR-Cas system. Genes Dev. 2016 Feb. 15; 30(4):447-59. doi: 10.1101/gad.272153.115. Epub 2016 Feb. 4. PMID: 26848045; PMCID: PMC4762429). Therefore, in the context of the present invention, the term rPAM designates the sequences flanking the RNA target that are required to activate CasQ. We also refer to CasΩ as Cas12a2.

Cas12 nucleases (within Type V CRISPR-Cas systems) are known to recognize and cleave DNA, thereby eliciting degradation of ssDNA. Since the development of the CRISPR-Cas9 system, various CRISPR systems have been identified in bacteria and archaea, including CRISPR from Prevotella and Francisella 1 (Cpf1, also known as Cas12a), and Cas14 (recently classified as Cas12f); these systems constitute a diverse genome editing toolbox in which each tool has unique utility. The CRISPR genome-editing tool consists of a gene-targeting guide RNA and a Cas endonuclease. These two components form a ribonucleoprotein (RNP) complex that recognizes target sequences accompanying a protospacer-adjacent motif (PAM), subsequently inducing a double-stranded break (DSB) either inside or outside the protospacer region.

U.S. Pat. No. 9,790,490B2 describes Cas12a (Cpf1) enzymes, including Cas12a (type V), which corresponds to CasΩ according to the present invention.

Recently, it was found that Cas12a also degrades non-specific single-stranded DNA (ssDNA) upon crRNA-mediated, specific, binding of either ssDNA or dsDNA. More recently, FRET and cryo-EM experiments demonstrated that Cas12a undergoes a series of checkpoints during target binding that culminates in exposure of the RuvC domain, which initially cleaves the unwound dsDNA target by first cutting the non-target strand, then the target strand, and subsequently remains activated, allowing for indiscriminate ssDNA cleavage (Swarts D C, Jinek M. Mechanistic Insights into the cis- and trans-Acting DNase Activities of Cas12a. Mol Cell. 2019 Feb. 7; 73(3):589-600.e4. doi: 10.1016/j.molcel.2018.11.021. Epub 2019 Jan. 10. PMID: 30639240; PMCID: PMC6858279). US20200399697A1 describes the diagnostic use of Cas12a based on its collateral degradation of ssDNA.

Smith C W, et al. (in: Probing CRISPR-Cas12a Nuclease Activity Using Double-Stranded DNA-Templated Fluorescent Substrates. Biochemistry. 2020 Apr. 21; 59(15):1474-1481. doi: 10.1021/acs.biochem.0c00140. Epub 2020 Apr. 7. PMID: 32233423; PMCID: PMC7384386) report a dsDNA substrate (probe-full) for probing Cas12a trans-cleavage activity upon target detection. A diverse set of Cas12a substrates with alternating dsDNA character were designed and studied using fluorescence spectroscopy. They observed that probe-full without any nick displayed trans-cleavage performance that was better than that of the form that contains a nick. Different experimental conditions of salt concentration, target concentration, and mismatch tolerance were examined to evaluate the probe performance. The activity of Cas12a was programmed for a dsDNA frame copied from a tobacco curly shoot virus (TCSV) or hepatitis B virus (HepBV) genome by using crRNA against TCSV or HepBV, respectively. While on-target activity offered detection of as little as 10 pM dsDNA target, off-target activity was not observed even at 1 nM control DNAs. They demonstrated that trans-cleavage of Cas12a is not limited to ssDNA substrates, and Cas12a-based diagnostics can be extended to dsDNA substrates.

U.S. Ser. No. 10/337,051B2, U.S. Ser. No. 10/494,664B2, U.S. Ser. No. 10/266,887B2, and US20180340219A1 disclose Cas13a (C2c2) as an RNA-targeting nuclease with collateral RNase activity, systems and methods for diagnostic use.

Baisong, T., et al. (in: The Versatile Type V CRISPR Effectors and Their Application Prospects, Frontiers in Cell and Developmental Biology, Vol. 8, 2021, p. 1835, DOI: 10.3389/fcell.2020.622103) disclose that the class 2 clustered regularly interspaced short palindromic repeats (CRISPR)-Cas systems, characterized by a single effector protein, can be further subdivided into types II, V, and VI. The application of the type II CRISPR effector protein Cas9 as a sequence-specific nuclease in gene editing has revolutionized the field of DNA manipulation. Similarly, Cas13 as the effector protein of type VI provides a convenient tool for RNA manipulation. Additionally, the type V CRISPR-Cas system is another valuable resource with many subtypes and diverse functions. In their review, they summarize all the subtypes of the type V family that have been identified so far. According to the functions currently displayed by the type V family, they attempt to introduce the functional principle, current application status, and development prospects in biotechnology for all major members.

It is an object of the present invention to provide an additional tool stemming from the above for the field of molecular diagnostics, as well as gene editing and therapy. Other objects and advantages will become apparent upon further studying the present specification with reference to the accompanying examples.

In a first aspect thereof, the object of the present invention is solved by providing a method for cleaving a nucleic acid molecule selected from dsDNA, ssDNA, and RNA, comprising the steps of a) providing at least one CasΩ nuclease enzyme, b) providing at least one preselected guide RNA, c) forming a complex between the least one CasΩ nuclease enzyme and the at least one preselected guide RNA, d) binding of the complex of c) to a target RNA based on the at least one preselected guide RNA, and e) cleaving said nucleic acid molecule selected from dsDNA, ssDNA, and RNA by the at least one CasΩ nuclease enzyme, wherein said at least one preselected guide RNA comprises a guide sequence that is at least 90% complementary to the target RNA.

In a second aspect thereof, the object of the present invention is solved by providing a complex comprising a CasΩ nuclease and at least one pre-selected guide RNA designed for binding to at least one target RNA. Preferred is the complex according to the present invention which further bound to a target RNA molecule having a guide sequence that is at least 90% complementary to said guide RNA, and wherein said target RNA is preferably flanked by at least one RNA protospacer adjacent motif (rPAM). In one embodiment, the rPAM preferably flanks the 3′ end of the target and is an A-rich sequence. In another embodiment, the rPAM is 5′-BAAA-3′.

In a third aspect thereof, the object of the present invention is solved by providing a method for detecting at least one target RNA in a cell, tissue, cellular nucleus, and/or sample, said method comprising a) providing at least one ssDNA, dsDNA or RNA reporter nucleic acid in said cell, tissue, cellular nucleus, and/or sample, b) contacting said cell, tissue, cellular nucleus, and/or sample with at least one complex between at least one CasΩ nuclease enzyme and at least one preselected guide RNA, preferably according to the present invention as above, wherein said at least one preselected guide RNA comprises a guide sequence that is at least 90% complementary to the target RNA, and c) detecting a cleaving, cutting and/or nicking of said at least one ssDNA, dsDNA or RNA reporter nucleic acid, wherein detecting said cleaving the at least one reporter nucleic acid detects said at least one target RNA in said cell, tissue, cellular nucleus and/or sample.

In a fourth aspect thereof, the object of the present invention is solved by providing a method for modulating expression of at least one target RNA in a cell, tissue, cellular nucleus, and/or sample, wherein said at least one target RNA is selected from an mRNA, non-coding RNA and a viral RNA molecule, said method comprising: a) contacting said cell, tissue, cellular nucleus, and/or sample with b) at least one complex between at least one CasΩ nuclease enzyme and at least one preselected guide RNA, preferably according to the present invention as above, wherein said at least one preselected guide RNA comprises a guide sequence that is at least 90% complementary to the at least one target RNA, and c) binding the complex of b) to the at least one target RNA and thereby altering the stability, processing, or translation of the at least one target RNA, whereby the binding in c) modulates the expression of at least one target RNA in the cell, tissue, cellular nucleus, and/or sample.

In a fifth aspect thereof, the object of the present invention is solved by providing a method for editing the sequence of at least one target RNA in a cell, tissue, cellular nucleus, and/or sample, wherein said at least one target RNA is selected from an mRNA, non-coding RNA and a viral RNA molecule, said method comprising: a) contacting said cell, tissue, cellular nucleus, and/or sample with b) at least one complex between at least one modified and catalytically inactive CasΩ nuclease enzyme complexed with at least one RNA-modifying enzyme, preferably according to the present invention as above, and at least one preselected guide RNA, wherein said at least one preselected guide RNA comprises a guide sequence that is at least 90% complementary to the at least one target RNA, and c) binding the complex of b) to the at least one target RNA and editing of the at least one target RNA by said at least one RNA-modifying enzyme.

In a sixth aspect thereof, the object of the present invention is solved by providing the complex according to the present invention for use in the prevention and/or treatment of diseases, such as for example, for use in the prevention and/or treatment of infections and/or genetic disorder, such as proliferative disorders, such as cancer, fungal, protozoan, bacterial and/or viral infections.

In a seventh aspect thereof, the object of the present invention is solved by providing a method for specifically inactivating an undesired cell, comprising contacting said cell with a complex according to the present invention, wherein said guide RNA is specifically selected for said undesired cell to be inactivated. The method can be preferably used to select for cells that remain unedited using the method according to the present invention.

In an eighth aspect thereof, the object of the present invention is solved by providing a method for preventing and/or treating a disease, such as for example, an infection and/or genetic disorder, such as a proliferative disorder, such as cancer, fungal, protozoan, bacterial and/or viral infections, an autoimmune disease, comprising administering to a subject in need of such treatment an effective amount of the complex according to the present invention.

In a ninth aspect thereof, the object of the present invention is solved by providing a method for decontaminating a preparation from an undesired contaminant, such as fungal, protozoan, bacterial and/or viral contamination, comprising suitably administering to said preparation an effective amount of the complex according to the present invention, and thereby removing and/or reducing the undesired contaminant.

In a tenth aspect thereof, the object of the present invention is solved by providing a use of the complex according to the present invention for cleaving a nucleic acid molecule selected from dsDNA, ssDNA, and RNA, for detecting at least one target RNA in a cell, tissue, cellular nucleus, and/or sample, for modulating expression of at least one target RNA in a cell, tissue, cellular nucleus, and/or sample, for editing the sequence of at least one target RNA in a cell, tissue, cellular nucleus, and/or sample, for specifically inactivating an undesired cell or virus, or for decontaminating a preparation from an undesired contaminant.

As mentioned above, in a first aspect thereof, the object of the present invention is solved by providing a method for cleaving a nucleic acid molecule selected from dsDNA, ssDNA, and RNA, comprising the steps of a) providing at least one CasΩ nuclease enzyme, b) providing at least one preselected guide RNA, c) forming a complex between the least one CasΩ nuclease enzyme and the at least one preselected guide RNA, d) binding of the complex of c) to a target RNA based on the at least one preselected guide RNA, and e) cleaving said nucleic acid molecule selected from dsDNA, ssDNA, and RNA by the at least one CasΩ nuclease enzyme. Preferably, the at least one preselected guide RNA comprises a guide sequence that is at least 90% complementary to the target RNA.

The present invention is based on the detection of an RNA target sequence by a CRISPR nuclease designated herein as CasΩ (and also Cas12a2) that utilizes a guide RNA to recognize complementary RNA sequence(s) flanked by an RNA PAM (rPAM) leading to the non-specific degradation (cleaving, cutting or nicking) of nucleic acids, including single-stranded DNA (ssDNA), double stranded DNA (dsDNA), and RNA. Given the structural similarity to the established Cas nuclease Cas12a, CasΩ was presumed to target DNA.

The combination of RNA recognition and triggered collateral ssDNA, dsDNA, and RNA degradation as well as the recognition of an rPAM is unique amongst known Cas nucleases. It offers a clear advantage over other Cas nucleases used for molecular diagnostics, offers a unique means of achieving RNA interference and RNA editing, and opens first applications in sequence-specific counterselection and killing of bacteria, archaea, and eukaryotes, as well as clearance of DNA and RNA viruses.

Zetsche B, et al. (in: Cpf1 is a single RNA-guided endonuclease of a class 2 CRISPR-Cas system. Cell. 2015 Oct. 22; 163(3):759-71. doi: 10.1016/j.cell.2015.09.038. Epub 2015 Sep. 25. PMID: 26422227; PMCID: PMC4638220) report characterization of Cpf1, a putative class 2 CRISPR effector. They demonstrate that Cpf1 mediates robust DNA interference with features distinct from Cas9. Cpf1 is a single RNA-guided endonuclease lacking tracrRNA, and it utilizes a T-rich protospacer-adjacent motif. Moreover, Cpf1 cleaves DNA via a staggered DNA double-stranded break. Out of 16 Cpf1-family proteins, they identified two candidate enzymes from Acidaminococcus and Lachnospiraceae with efficient genome-editing activity in human cells. A CasΩ enzyme is disclosed from Sulfuricurvum_sp_PC₀₈₋₆₆.

Begemann, M. B., et al. (in: Characterization and validation of a novel group of Type V, Class 2 nucleases for in vivo genome editing. 2017. bioRxiv, pp. 1-9) present some evidence of enzymes and genome editing in plants. According to the results as produced in the context of the present inventors, it seems that the genomic deletions as observed were not due to DNA targeting, but rather resulted from the purifying selection in response to RNA targeting.

Makarova, K. S., et al. (in: Classification and Nomenclature of CRISPR-Cas Systems: Where from Here? 2018. The CRISPR journal, 1(5), pp. 325-336) disclose a CasΩ from the Sm clade (KF067988.1) as a Cas12a variant, which was grouped with two other nucleases that seem not to be CasΩ.

Aliaga Goltsman, D. S. et al. (in: Novel Type V-A CRISPR Effectors Are Active Nucleases with Expanded Targeting Capabilities. 2020. The CRISPR journal, 3(6), pp. 454-461) classified a number of CasΩ nucleases from the Sm clade as Cas12a, namely Cas12a-M60-3, Cas12a-M60-1, Cas12a-M60-8, Cas12a-M60-9, Cas12a-M26-5, Cas12a-M26-14, and Cas12a-M26-15.

US 2019/0048357, which is incorporated by reference in its entirety, discloses a method of modifying a nucleotide sequence at a target site in the genome of a eukaryotic cell, preferably a plant cell. For this, a Cms1 polypeptide, or a polynucleotide encoding a Cms1 polypeptide and a DNA-targeting RNA, or a DNA polynucleotide encoding a DNA-targeting RNA, wherein the DNA-targeting RNA comprises: (a) a first segment comprising a nucleotide sequence that is complementary to a sequence in the target DNA; and (b) a second segment that interacts with a Cms1 polypeptide are introduced into the cell. The method requires to then modify said nucleotide sequence at said target site, and wherein said genome of a eukaryotic cell is a nuclear, plastid, or mitochondrial genome.

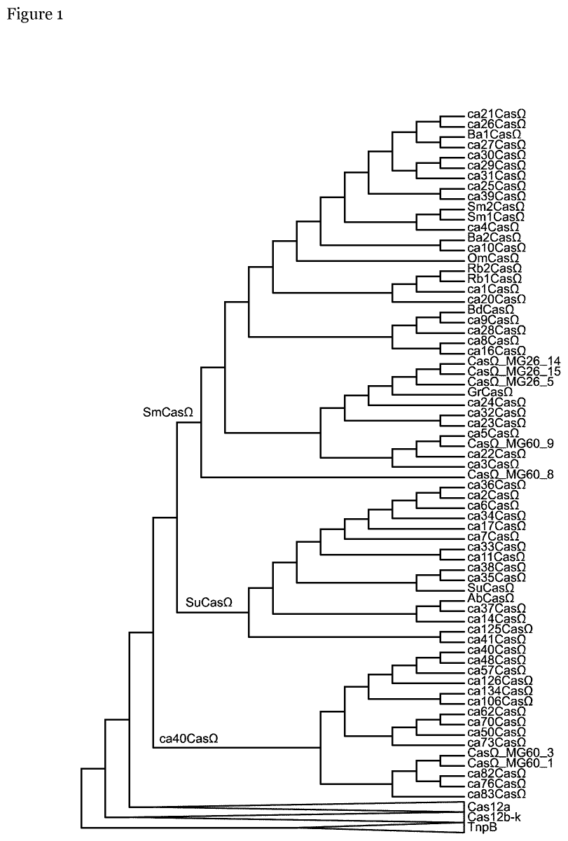

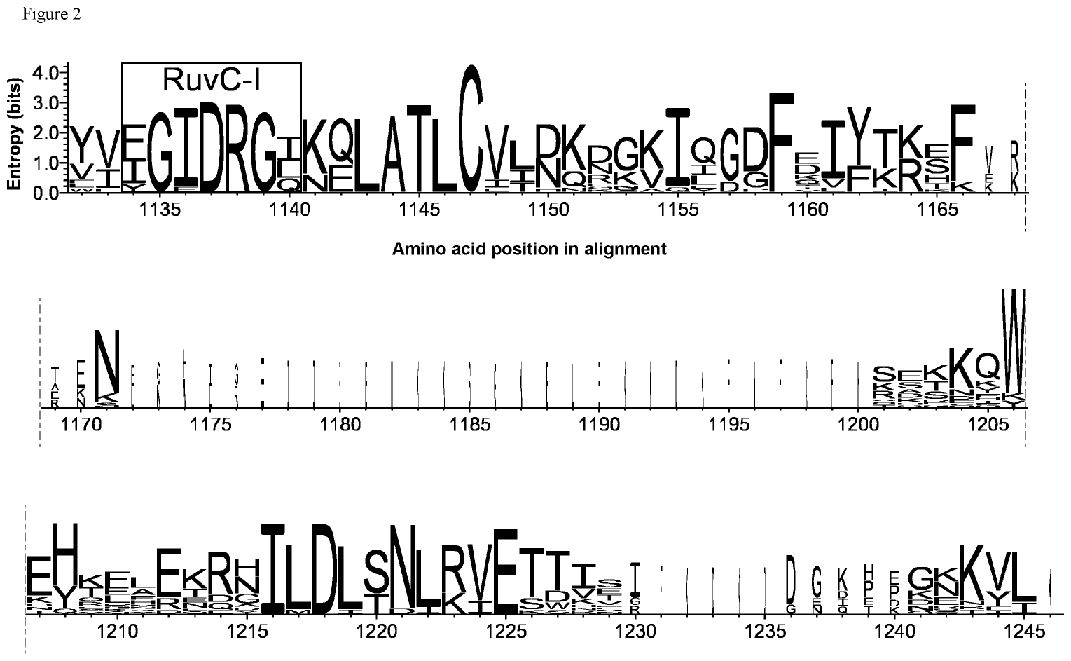

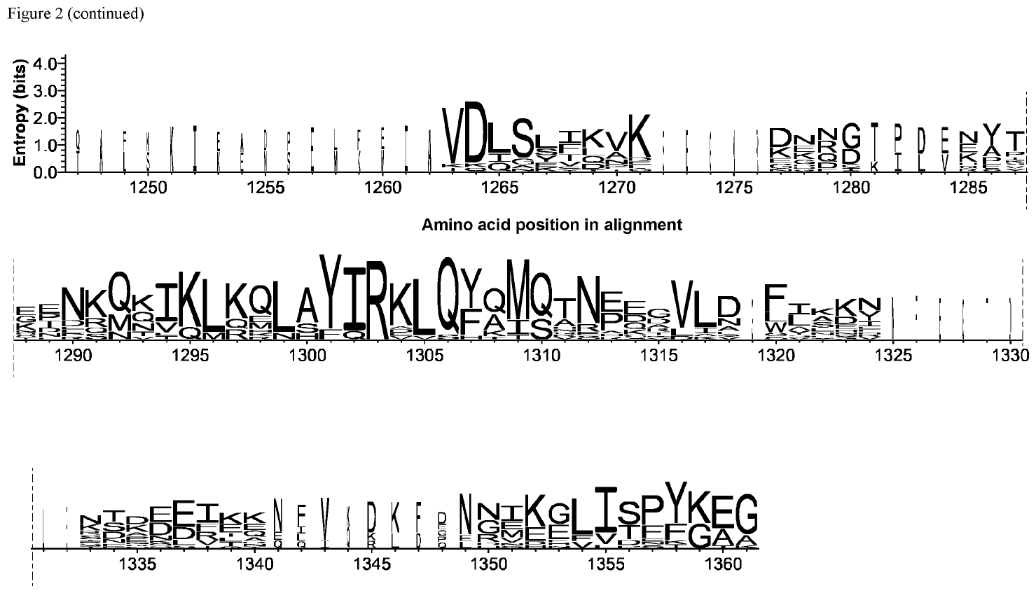

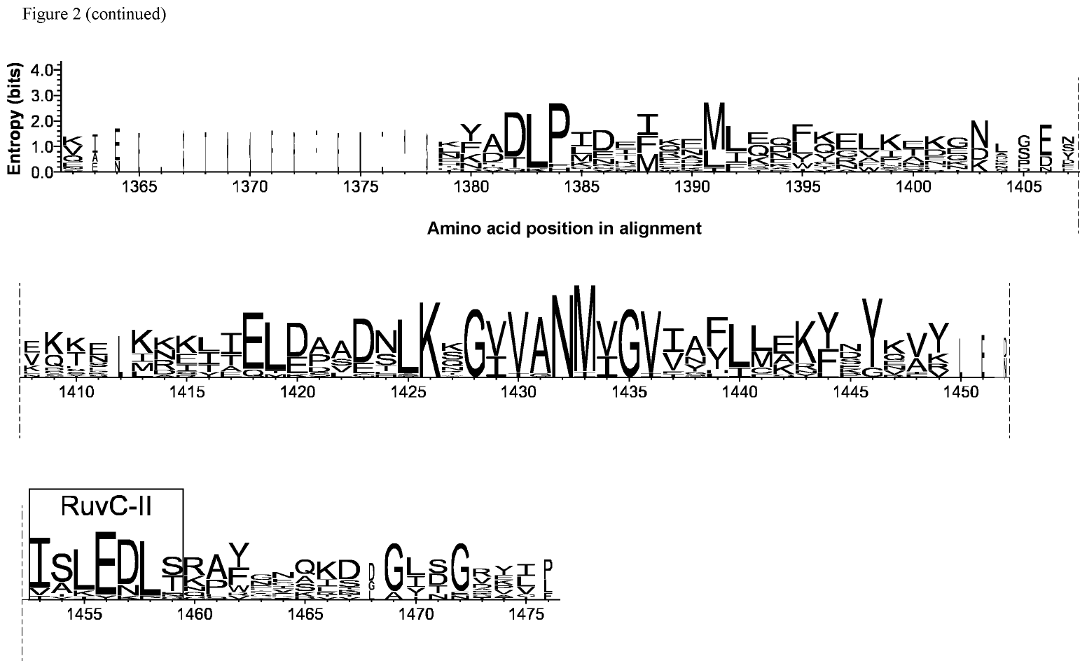

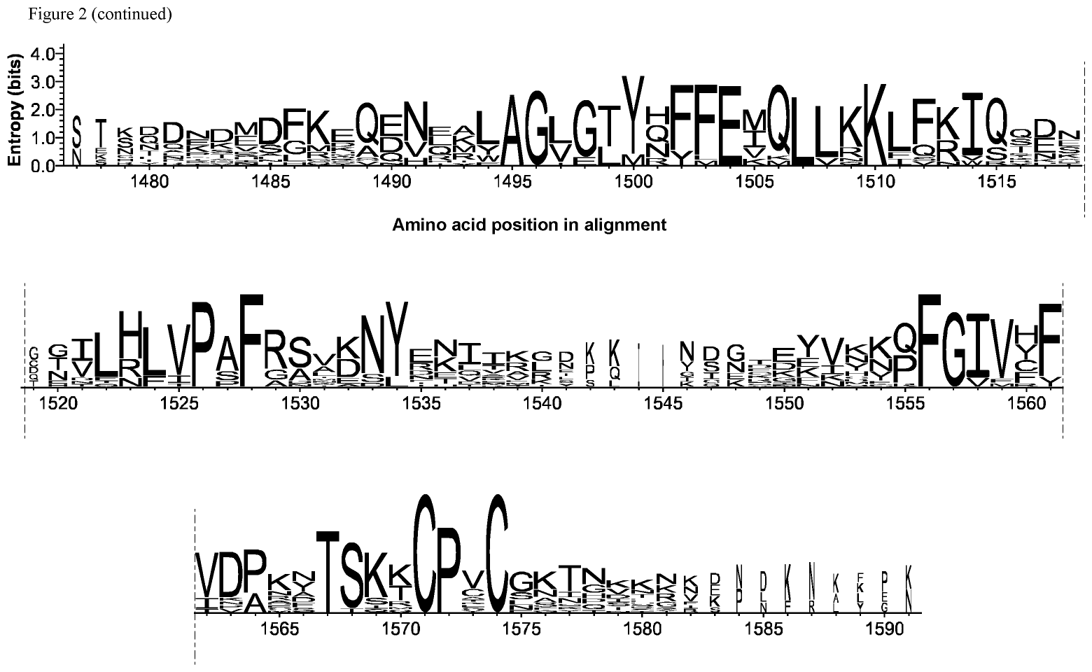

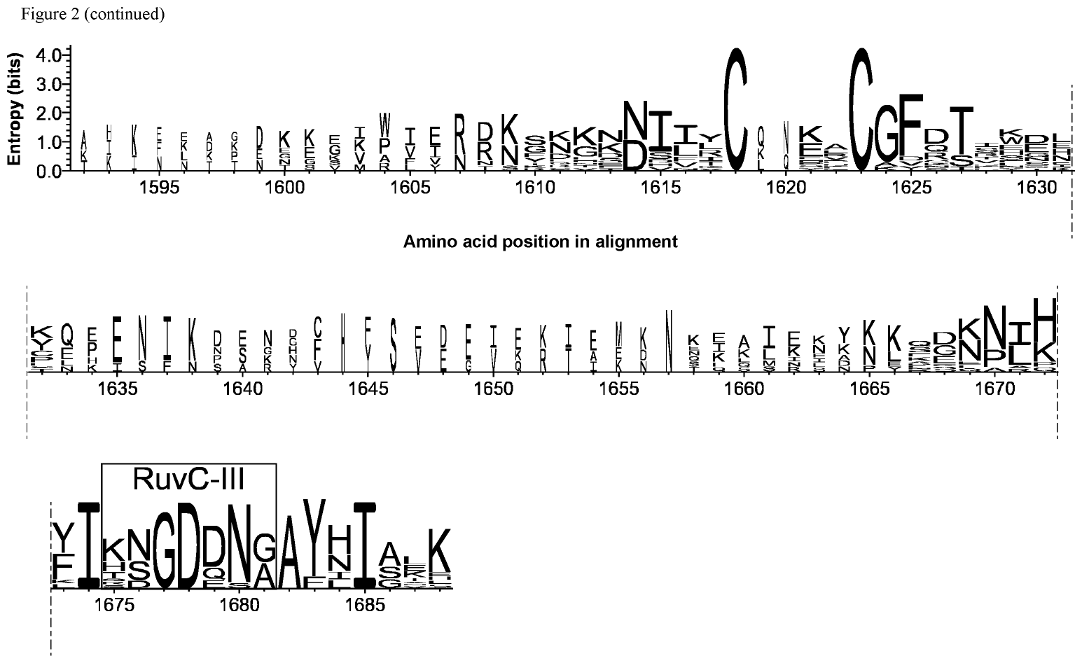

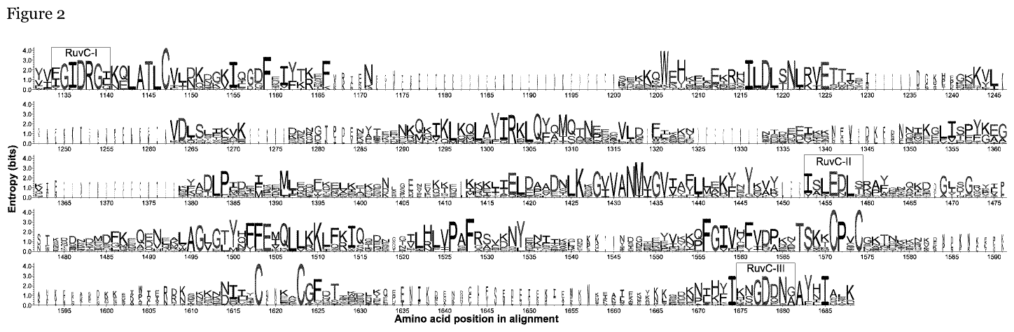

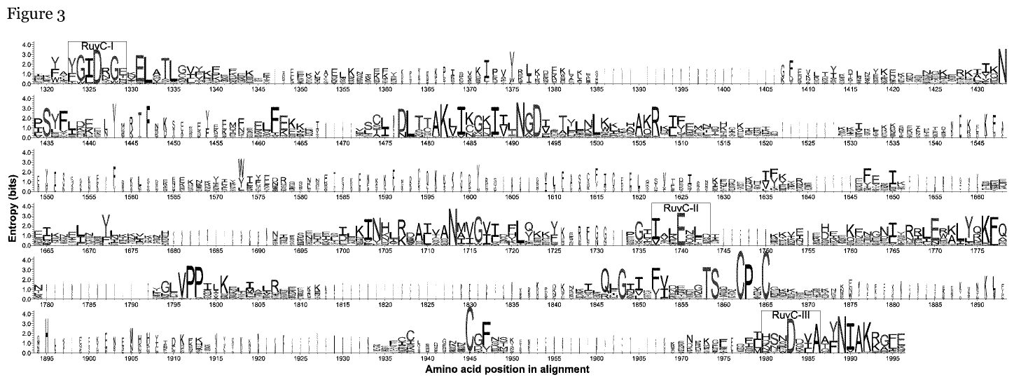

FIG. 1 of US 2019/0048357 shows a phylogenetic tree drawn from a RuvC-anchored MUSCLE alignment of the Type V nuclease amino acid sequences indicated. Sm-type, Sulf-type, and Unk40-type Cms1 nucleases are indicated. FIG. 2 then shows a summary of amino acid motifs shared among Sm-type Cms1 proteins. The weblogo figures in boxes 1-10 correspond to SEQ ID NOs: 177-186 of US 2019/0048357, respectively, and their locations on the SmCms1 protein (SEQ ID NO: 10 of US 2019/0048357) are shown. FIG. 3 shows a summary of amino acid motifs shared among Sulf-type Cms1 proteins. The weblogo figures in boxes 1-17 correspond to SEQ ID NOs: 288-289 and SEQ ID NOs: 187-201 of US 2019/0048357, respectively, and their locations on the SulfCms1 protein (SEQ ID NO: 11 of US 2019/0048357) are shown.

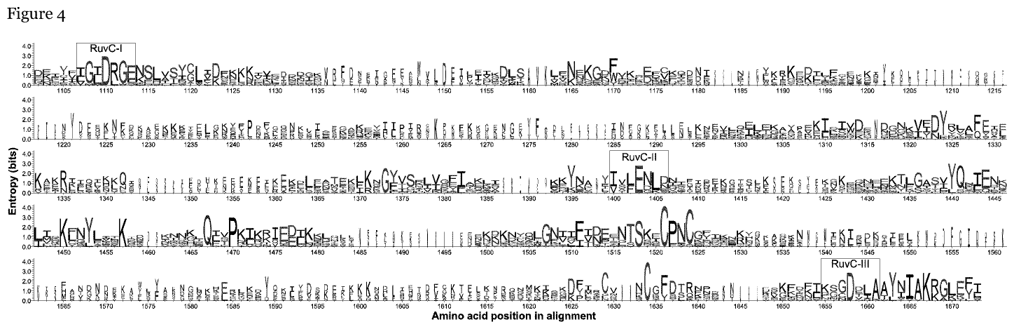

FIG. 4 shows a summary of amino acid motifs shared among Unk40-type Cms1 proteins. The weblogo figures in boxes 1-7 correspond to SEQ ID NOs: 290-296, respectively, and their locations on the Unk40Cms1 protein (SEQ ID NO:68) are shown.

Therefore, US 2019/0048357 in form of the Sm-type Cms1 proteins and the Sulf-type Cms1 proteins as well as the Unk40-type Cms1 discloses preferred examples of CasΩ nucleases according to the present invention. In the context of the present invention, the term CasΩ nuclease or CasΩ nuclease enzyme shall therefore include Cas nuclease polypeptides or respective functional fragments thereof that exhibit at least the following features;
a) CRISPR-associated single-effector nuclease enzyme with a RuvC domain consisting of at least one RuvC motif, more preferably two RuvC motifs, more preferably three RuvC motifs, and preferably no HNH or HEPN domains,
b) Unique amino acid composition between the RuvC-I and RuvC-II motifs, comprising an insertion of amino acids of one of three motifs compared with non-CasΩ nucleases,
c) Unique amino acid composition between the RuvC-II and RuvC-III motifs, comprising a deletion of amino acids compared with non-CasΩ nucleases, and replaced with a Zn-finger domain,
d) Ability of the nuclease to process CRISPR RNA repeat without accessory factors (i.e., without tracrRNA and/or RNase III),
e) Nuclease recognizes single-stranded RNA as its unique nucleic-acid target,
f) Nuclease naturally targets RNA flanked by an rPAM, and
f) Recognition of the RNA leads to non-specific (non-sequence specific) cleavage of ssRNA, ssDNA, and/or dsDNA.

In the context of the present invention, the term CasΩ nuclease or CasΩ nuclease enzyme shall also include polypeptides having at least 50%, preferably at least 70, more preferably at least 80%, more preferably at least 90%, and more preferably at least 95% identity with a sequence selected from the group consisting of SEQ ID NOs: 10 or 11 or 68 as disclosed in US 2019/0048357, and having RNA-dependent CasΩ nuclease activity, i.e., non-specifically cleaving dsDNA, ssDNA, and/or RNA.

Preferred is a CasΩ nuclease or CasΩ nuclease enzyme of the Su-clade of enzymes (see FIG. 1), which thus includes the polypeptides having at least 80%, more preferably at least 90%, and more preferably at least 95% identity with the amino acid sequence according to SEQ ID NOs: 11 as disclosed in US 2019/0048357, and having RNA-dependent CasΩ nuclease activity, i.e., non-specifically cleaving dsDNA, ssDNA, and/or RNA.

CasΩ nuclease amino acid sequence alignments were examined to identify motifs within the protein sequences that are well-conserved among these nucleases. It was observed that CasΩ nucleases were found in three well-separated clades on the phylogenetic tree shown in FIG. 1. One of these clades includes Sm CasΩ (SEQ ID NO: 10 as disclosed in US 2019/0048357), another includes Su CasΩ (SEQ ID NO: 11 as disclosed in US 2019/0048357), and the third includes Unk40 (SEQ ID NO: 68 as disclosed in US 2019/0048357). Members of each of these clades were therefore aligned separately to identify partially and/or completely conserved amino acid motifs among these nucleases. For the alignment of Sm CasΩ nucleases, SEQ ID NOs: 10, 20, 23, 30, 32-34, 37-39, 41, 43, 44, 46-60, 67, 154-156, 208-211, 222, 223, 225, 228, 229, 232, 234, 236, 237, 241, 243, 245, 248, 250, 251, 253, and 254 as disclosed in US 2019/0048357 were aligned. For the alignment of Su CasΩ nucleases, SEQ ID NOs: 11, 21, 22, 31, 35, 36, 40, 42, 45, 61-66, 69, 227, 230, 231, 235, 239, 240, 242, 244, and 247 as disclosed in US 2019/0048357 were aligned. For the alignment of Unk40 CasΩ nucleases, SEQ ID NOs: 68, 224, 226, 233, 238, 246, 249, and 252 were aligned. These alignments were performed in US 2019/0048357 using MUSCLE and the resulting alignments were examined manually to identify regions that showed conservation among all of the aligned proteins.

The amino acid (motifs) shown in present SEQ ID NOs: 32 to 67 were identified from the alignment of Sm CasΩ nucleases; the amino acid motifs shown in present SEQ ID NOs: 16 to 31 were identified from the alignment of Su CasΩ nucleases; the amino acid motifs shown in present SEQ ID NOs: 1 to 15 were identified from the alignment of ca40 (Unk40) CasΩ nucleases. Schematic diagrams showing the locations of these conserved motifs on the Sm CasΩ, and Su CasΩ protein sequences are presented in FIGS. 2 to 4.

The nucleases according to the present invention can also be distinguished/grouped based on the following (additional) features. The particularly preferred subgroup of SuCasΩ nucleases according to the present invention, in particular as shown in SEQ ID Nos. 16 to 31, as one distinguishing feature exhibits a unique amino acid composition between the RuvC-II and RuvC-III catalytic motifs, comprising a deletion of amino acids compared to non-CasΩ nucleases, such as Cas12a (see also FIG. 2). The subgroup of SmCasΩ nucleases according to the present invention, in particular as shown in SEQ ID Nos. 32 to 67, as one distinguishing feature exhibits a unique amino acid composition between the RuvC-II and RuvC-III catalytic motifs, comprising a replacement of amino acids with a Zn-finger domain compared to non-CasΩ nucleases, such as Cas12a. Finally, the subgroup of ca40CasΩ nucleases according to the present invention, in particular as shown in SEQ ID Nos. 1 to 15, as one distinguishing feature exhibits a unique amino acid composition between the RuvC-II and RuvC-III catalytic motifs, comprising a replacement of amino acids with a Zn-finger domain compared to non-Cas2 nucleases, such as Cas12a.

Particularly preferred CasΩ nuclease enzymes according to and to be used in accordance with the present invention have been identified as shown in the following table.

| CasΩ-clade/ | | | |
|---|---|---|---|
| SEQ ID NO: | Organism (if known) | Enzyme name, Accession number | notes |
| ca40 | Unknown | ca106CasΩ, Ga0232645_1000961 | SEQ 68 of US |
| (Unk40)/1 | | | 2019/0048357 |
| ca40 | Unknown | ca134CasΩ, Ga0451652_001661 | |
| (Unk40)/2 | | | |
| ca40 | Unknown | ca126CasΩ, Ga0153773_1000694 | |
| (Unk40)/3 | | | |
| ca40 | Unknown | ca40CasΩ, Ga0180009_10008218 | SEQ 300 of US |
| (Unk40)/4 | | | 2019/0048357 |
| ca40 | Unknown | ca48CasΩ, Ga0066649_10040698, | SEQ 224 of US |
| (Unk40)/5 | | Ga0066640_10036548 | 2019/0048357 |
| ca40 | Unknown | ca57CasΩ, Ace Lake Antarctica | SEQ 223 of US |
| (Unk40)/6 | | sample | 2019/0048357 |
| ca40 | Unknown | ca73CasΩ, Ga0209097_10004342 | SEQ 249 of US |
| (Unk40)/7 | | | 2019/0048357 |
| ca40 | Unknown | ca50CasΩ, | SEQ 226 of US |
| (Unk40)/8 | | TB_GS09_5DRAFT_1000006 | 2019/0048357 |
| ca40 | Unknown | ca62CasΩ, Ga0103269_100007 | SEQ 238 of US |
| (Unk40)/9 | | | 2019/0048357 |
| ca40 | Unknown | ca70CasΩ, Ga0209718_1004048 | SEQ 246 of US |
| (Unk40)/10 | | | 2019/0048357 |
| ca40 | Unknown | ca83CasΩ, Ga0256843_1010138 | |
| (Unk40)/11 | | | |
| ca40 | Unknown | ca76CasΩ, Ga0256840_1005709 | SEQ 252 of US |
| (Unk40)/12 | | | 2019/0048357 |
| ca40 | Unknown | ca82CasΩ, Ga0256834_1002422 | SEQ 23 of US |
| (Unk40)/13 | | | 2019/0048357 |
| ca40 | Unknown | CasΩ_MG60_1, Goltsman et al., | |
| (Unk40)/14 | | 2020 | |
| ca40 | Unknown | CasΩ_MG60_3, Goltsman et al., | |
| (Unk40)/15 | | 2020 | |
| Su CasΩ/16 | Unknown | ca37CasΩ, Ga0031101 | SEQ 65 of US |
| | | | 2019/0048357 |
| Su CasΩ/17 | Absconditabacteria | AbCasΩ, JABCPD020000008 | |
| Su CasΩ/18 | Unknown | ca14CasΩ, Ga0031091 | SEQ 42 of US |
| | | | 2019/0048357 |
| Su CasΩ/19 | Unknown | ca11CasΩ, Ga0003449 | SEQ 40 of US |
| | | | 2019/0048357 |
| Su CasΩ/20 | Unknown | ca33CasΩ, Ga0099364_10008519 | SEQ 61 of US |
| | | | 2019/0048357 |
| Su CasΩ/21 | Sulfuricurvum sp. PC08-66 | SuCasΩ, JQIT00000000.1 | SEQ 11 of US |
| | | | 2019/0048357 |
| Su CasΩ/22 | Unknown | ca35CasΩ, Ga0099364_10008519 | SEQ 63 of US |
| | | | 2019/0048357 |
| Su CasΩ/23 | Unknown | ca38CasΩ, Ga0026479 | SEQ 66 of US |
| | | | 2019/0048357 |
| Su CasΩ/24 | Unknown | ca7CasΩ, Ga0079224_1000149946 | SEQ 36 of US |
| | | | 2019/0048357 |
| Su CasΩ/25 | Unknown | ca6CasΩ, Ga0079223 | SEQ 35 of US |
| | | | 2019/0048357 |
| Su CasΩ/26 | Unknown | ca17CasΩ, Ga0123357_10004810 | SEQ 45 of US |
| | | | 2019/0048357 |
| Su CasΩ/27 | Unknown | ca34CasΩ, Ga0005846 | SEQ 62 of US |
| | | | 2019/0048357 |
| Su CasΩ/28 | Unknown | ca2CasΩ, Ga0025131 | SEQ 31 of US |
| | | | 2019/0048357 |
| Su CasΩ/29 | Unknown | ca36CasΩ, Ga0160425_10032006 | SEQ 64 of US |
| | | | 2019/0048357 |
| Su CasΩ/30 | Unknown | ca41CasΩ, Ga0066604_10155245 | SEQ 69 of US |
| | | and Ga0066603_10011461 and | 2019/0048357 |
| | | Ga0066603_10343056 | |
| Su CasΩ/31 | Unknown | ca125CasΩ, Ga0153773_1000694 | |
| Sm CasΩ/32 | Unknown | CasΩ_MG60_8, Goltsman et al., | |
| | | 2020 | |
| Sm CasΩ/33 | Unknown | ca24CasΩ, KVWGV2_combined | SEQ 52 of US |
| | | | 2019/0048357 |
| Sm CasΩ/34 | Gracilibacteria bacterium | GrCasΩ, RAL57497.1 | |
| | GN02-872 GN02_872_C01 | | |
| Sm CasΩ/35 | Unknown | CasΩ_MG26_5, Goltsman et al., | |
| | | 2020 | |
| Sm CasΩ/36 | Unknown | CasΩ_MG26_14, Goltsman et al., | |
| | | 2020 | |
| Sm CasΩ/37 | Unknown | CasΩ_MG26_15, Goltsman et al., | |
| | | 2020 | |
| Sm CasΩ/38 | Unknown | ca23CasΩ, Ga0069611 | SEQ 51 of US |
| | | | 2019/0048357 |
| Sm CasΩ/39 | Unknown | ca32CasΩ, Ga0031051 | SEQ 60 of US |
| | | | 2019/0048357 |
| Sm CasΩ/40 | Unknown | ca3CasΩ, Ga0070389 | SEQ 32 of US |
| | | | 2019/0048357 |
| Sm CasΩ/41 | Unknown | ca22CasΩ, Ga0172377_10049484 | SEQ 50 of US |
| | | | 2019/0048357 |
| Sm CasΩ/42 | Unknown | ca5CasΩ, Ga0074197 | SEQ 34 of US |
| | | | 2019/0048357 |
| Sm CasΩ/43 | Unknown | CasΩ_MG60_9, Goltsman et al., | |
| | | 2020 | |
| Sm CasΩ/44 | Unknown | ca28CasΩ, Ga0114934 | SEQ 56 of US |
| | | | 2019/0048357 |
| Sm CasΩ/45 | Bdellovibrionales bacterium | BdCasΩ, Ga0325881_105 | |
| | SP5DBV1 | | |
| Sm CasΩ/46 | Unknown | ca9CasΩ, Ga0116195 | SEQ 38 of US |
| | | | 2019/0048357 |
| Sm CasΩ/47 | Unknown | ca8CasΩ, Ga0116185 | SEQ 37 of US |
| | | | 2019/0048357 |
| Sm CasΩ/48 | Unknown | cal6CasΩ, a0066637 | SEQ 44 of US |
| | | | 2019/0048357 |
| Sm CasΩ/49 | Unknown | ca20CasΩ, Ga0172381_10024559 | SEQ 48 of US |
| | | | 2019/0048357 |
| Sm CasΩ/50 | Unknown | ca1CasΩ, Ga0066603 | SEQ 30 of US |
| | | | 2019/0048357 |
| Sm CasΩ/51 | Roizmanbacteria bacterium | Rb1CasΩ, LBTJ01000016 | SEQ 154 of US |
| | GW2011_GWA2_37_7 | | 2019/0048357 |
| | US54_C0016 | | |
| Sm CasΩ/52 | Roizmanbacteria bacterium | Rb2CasΩ, Ga0301324_1082 | |
| | CG | | |
| Sm CasΩ/53 | Unknown | ca25CasΩ, Ga0123348 and | SEQ 54 of US |
| | | Ga0123349 | 2019/0048357 |
| Sm CasΩ/54 | Unknown | ca39CasΩ, Ga0187907_10021093 | SEQ 67 of US |
| | | | 2019/0048357 |
| Sm CasΩ/55 | Unknown | ca21CasΩ, Ga0005846 | SEQ 49 of US |
| | | | 2019/0048357 |
| Sm CasΩ/56 | Unknown | ca26CasΩ, Ga0082212_10027777 | SEQ 54 of US |
| | | | 2019/0048357 |
| Sm CasΩ/57 | Bacteroidetes bacterium | Ba1CasΩ, HDR01000103, | |
| | | PKP47251.1 | |
| Sm CasΩ/58 | Unknown | ca27CasΩ, Ga0003456 | SEQ 55 of US |
| | | | 2019/0048357 |
| Sm CasΩ/59 | Unknown | ca31CasΩ, Ga003455 | SEQ 59 of US |
| | | | 2019/0048357 |
| Sm CasΩ/60 | Unknown | ca29CasΩ, Ga0123353_10032784 | SEQ 57 of US |
| | | | 2019/0048357 |
| Sm CasΩ/61 | Unknown | ca30CasΩ, Ga0005842 | SEQ 58 of US |
| | | | 2019/0048357 |
| Sm CasΩ/62 | Omnitrophica | OmCasΩ, Ga0156434_161 | SEQ 155 of US |
| | WOR_2 bacterium | | 2019/0048357 |
| | RIFOXYA2_FULL_38_17 | | |
| Sm CasΩ/63 | Bacteroidetes bacterium | Ba2CasΩ, Ga0139412_1053 | |
| Sm CasΩ/64 | Unknown | ca10CasΩ, Ga0012990 | SEQ 39 of US |
| | | | 2019/0048357 |
| Sm CasΩ/65 | Unknown | ca4CasΩ, Ga0070419_1441414 | SEQ 33 of US |
| | | | 2019/0048357 |
| Sm CasΩ/66 | Smithella sp. SCADC | Sm1CasΩ, Ga0057837_1120 | SEQ 10 of US |
| | | | 2019/0048357 |
| Sm CasΩ/67 | Smithella sp. SC_K08D17 | Sm2CasΩ, Ga0069545_1011 | SEQ 156 of US |
| | | | 2019/0048357 |

The positions of the RuvC motifs for the nucleases were identified as follows:

| CasΩ- | RuvC-I | RuvC-I | RuvC-II | RuvC-II | RuvC-III | RuvC-III |
|---|---|---|---|---|---|---|
| clade/SEQ | start | end | start | end | start | end |
| ID NO: | (aa) | (aa) | (aa) | (aa) | (aa) | (aa) |
| ca40 | 760 | 766 | 1015 | 1021 | 1156 | 1162 |
| (Unk40)/1 | | | | | | |
| ca40 | 708 | 714 | 965 | 971 | 1109 | 1115 |
| (Unk40)/2 | | | | | | |
| ca40 | 822 | 828 | 1035 | 1041 | N/A | N/A |
| (Unk40)/3 | | | | | | |
| ca40 | 725 | 731 | 935 | 941 | 1139 | 1145 |
| (Unk40)/4 | | | | | | |
| ca40 | 731 | 737 | 936 | 942 | 1137 | 1143 |
| (Unk40)/5 | | | | | | |
| ca40 | 746 | 752 | 957 | 963 | 1159 | 1165 |
| (Unk40)/6 | | | | | | |
| ca40 | 766 | 772 | 887 | 893 | 1100 | 1106 |
| (Unk40)/7 | | | | | | |
| ca40 | 696 | 702 | 881 | 887 | 1078 | 1084 |
| (Unk40)/8 | | | | | | |
| ca40 | 668 | 674 | 854 | 860 | 1061 | 1067 |
| (Unk40)/9 | | | | | | |
| ca40 | 669 | 675 | 855 | 861 | 1065 | 1071 |
| (Unk40)/10 | | | | | | |
| ca40 | 687 | 693 | 873 | 879 | 1014 | 1020 |
| (Unk40)/11 | | | | | | |
| ca40 | 788 | 794 | 998 | 1004 | 1172 | 1178 |
| (Unk40)/12 | | | | | | |
| ca40 | 763 | 769 | 984 | 990 | 1152 | 1158 |
| (Unk40)/13 | | | | | | |
| ca40 | 794 | 800 | 922 | 928 | 1074 | 1080 |
| (Unk40)/14 | | | | | | |
| ca40 | 797 | 803 | 931 | 937 | 1093 | 1099 |
| (Unk40)/15 | | | | | | |
| Su | 873 | 879 | 1084 | 1090 | 1280 | 1286 |
| CasΩ/16 | | | | | | |
| Su | 866 | 872 | 1077 | 1083 | 1271 | 1277 |
| CasΩ/17 | | | | | | |
| Su | 526 | 532 | 737 | 743 | 895 | 901 |
| CasΩ/18 | | | | | | |
| Su | 874 | 880 | 1071 | 1077 | 1219 | 1225 |
| CasΩ/19 | | | | | | |
| Su | 862 | 868 | 1062 | 1068 | 1235 | 1241 |
| CasΩ/20 | | | | | | |
| Su | 845 | 851 | 1060 | 1066 | 1210 | 1216 |
| CasΩ/21 | | | | | | |
| Su | 848 | 854 | 1063 | 1069 | 1254 | 1260 |
| CasΩ/22 | | | | | | |
| Su | 858 | 864 | 1073 | 1079 | 1264 | 1270 |
| CasΩ/23 | | | | | | |
| Su | 850 | 856 | 1056 | 1062 | 1217 | 1223 |
| CasΩ/24 | | | | | | |
| Su | 853 | 859 | 1076 | 1082 | 1229 | 1235 |
| CasΩ/25 | | | | | | |
| Su | 651 | 657 | 852 | 858 | 1007 | 1013 |
| CasΩ/26 | | | | | | |
| Su | 853 | 859 | 1076 | 1082 | 1248 | 1254 |
| CasΩ/27 | | | | | | |
| Su | 862 | 868 | 1089 | 1095 | 1260 | 1266 |
| CasΩ/28 | | | | | | |
| Su | 869 | 875 | 1095 | 1101 | 1269 | 1275 |
| CasΩ/29 | | | | | | |
| Su | 867 | 873 | 1150 | 1156 | 1319 | 1325 |
| CasΩ/30 | | | | | | |
| Su | 972 | 978 | 1260 | 1266 | N/A | N/A |
| CasΩ/31 | | | | | | |
| Sm | 822 | 828 | 1051 | 1057 | 1166 | 1172 |
| CasΩ/32 | | | | | | |
| Sm | 751 | 757 | 990 | 996 | 1142 | 1148 |
| CasΩ/33 | | | | | | |
| Sm | 683 | 689 | 926 | 932 | 1089 | 1095 |
| CasΩ/34 | | | | | | |
| Sm | 572 | 578 | 798 | 804 | 967 | 973 |
| CasΩ/35 | | | | | | |
| Sm | 703 | 709 | 931 | 937 | 1083 | 1089 |
| CasΩ/36 | | | | | | |
| Sm | 703 | 709 | 931 | 937 | 1083 | 1089 |
| CasΩ/37 | | | | | | |
| Sm | 758 | 764 | 973 | 979 | 1108 | 1114 |
| CasΩ/38 | | | | | | |
| Sm | 819 | 825 | 1030 | 1036 | 1179 | 1185 |
| CasΩ/39 | | | | | | |
| Sm | 724 | 730 | 948 | 954 | 1064 | 1070 |
| CasΩ/40 | | | | | | |
| Sm | 791 | 797 | 1022 | 1028 | 1143 | 1149 |
| CasΩ/41 | | | | | | |
| Sm | 738 | 744 | 973 | 979 | 1090 | 1096 |
| CasΩ/42 | | | | | | |
| Sm | 773 | 779 | 1012 | 1018 | 1145 | 1151 |
| CasΩ/43 | | | | | | |
| Sm | 766 | 772 | 1024 | 1030 | 1141 | 1147 |
| CasΩ/44 | | | | | | |
| Sm | 799 | 805 | 1047 | 1053 | 1195 | 1201 |
| CasΩ/45 | | | | | | |
| Sm | 699 | 705 | 959 | 965 | 1087 | 1093 |
| CasΩ/46 | | | | | | |
| Sm | 675 | 681 | 912 | 918 | 1024 | 1030 |
| CasΩ/47 | | | | | | |
| Sm | 640 | 646 | 871 | 877 | 996 | 1002 |
| CasΩ/48 | | | | | | |
| Sm | 724 | 730 | 954 | 960 | 1066 | 1072 |
| CasΩ/49 | | | | | | |
| Sm | 767 | 773 | 1010 | 1016 | 1128 | 1134 |
| CasΩ/50 | | | | | | |
| Sm | 693 | 699 | 919 | 925 | 1037 | 1043 |
| CasΩ/51 | | | | | | |
| Sm | 692 | 698 | 919 | 925 | 1060 | 1066 |
| CasΩ/52 | | | | | | |
| Sm | 729 | 735 | 1046 | 1052 | 1146 | 1152 |
| CasΩ/53 | | | | | | |
| Sm | 733 | 739 | 1049 | 1055 | 1150 | 1156 |
| CasΩ/54 | | | | | | |
| Sm | 732 | 738 | 980 | 986 | 1085 | 1091 |
| CasΩ/55 | | | | | | |
| Sm | 723 | 729 | 1041 | 1047 | 1146 | 1152 |
| CasΩ/56 | | | | | | |
| Sm | 709 | 715 | 1029 | 1035 | 1134 | 1140 |
| CasΩ/57 | | | | | | |
| Sm | 729 | 735 | 1045 | 1051 | 1152 | 1158 |
| CasΩ/58 | | | | | | |
| Sm | 752 | 758 | 1069 | 1075 | 1170 | 1176 |
| CasΩ/59 | | | | | | |
| Sm | 719 | 725 | 1031 | 1037 | 1163 | 1169 |
| CasΩ/60 | | | | | | |
| Sm | 736 | 742 | 1052 | 1058 | 1163 | 1169 |
| CasΩ/61 | | | | | | |
| Sm | 724 | 730 | 936 | 942 | 1050 | 1056 |
| CasΩ/62 | | | | | | |
| Sm | 897 | 903 | 1177 | 1183 | 1321 | 1327 |
| CasΩ/63 | | | | | | |
| Sm | 669 | 675 | 893 | 899 | 1018 | 1024 |
| CasΩ/64 | | | | | | |
| Sm | 741 | 747 | 966 | 972 | 1088 | 1094 |
| CasΩ/65 | | | | | | |
| Sm | 698 | 704 | 919 | 925 | 1042 | 1048 |
| CasΩ/66 | | | | | | |
| Sm | 698 | 704 | 923 | 929 | 1063 | 1069 |
| CasΩ/67 | | | | | | |

The method according to the present invention then provides at least one preselected guide RNA designed for binding to at least one target RNA. Successful binding and recognition of the target RNA provides the signal that then leads to degradation of nucleic acids, such as DNA and RNA.

The parts of the nucleic acid molecules as used in the methods of the present invention that hybridize are complementary to at least 80%, preferably complementary to more than 90%, more preferably to more than 95%, and most preferably are 100% complementary to each other. Thus, the nucleotide sequence of said portion/part of said guide RNA that specifically hybridizes with the target RNA preferably excluding the rPAM can be produced and/or modified in order to be complementary to at least 80%, preferably complementary to more than 90%, more preferably to more than 95%, and most preferably are 100% complementary to the target RNA.

Certain portions of the nucleic acid molecules as used in the methods according to the present invention are found and/or designed to specifically hybridize with complementary portions in other molecules. As known to the person of skill, for this, the hybridization and washing conditions are critical. If the sequences are 100% complementary, then a high stringency hybridization may be carried out. Nevertheless, according to the invention, the portions that hybridize and/or specifically hybridize are complementary to at least 80%, preferably complementary to more than 90%, more preferably to more than 95%, and most preferably are 100% complementary. The stringency of hybridization is determined by the hybridization temperature and the salt concentration in the hybridization buffer, and high temperature and low salt is more stringent. A commonly used washing solution is SSC (Saline Sodium Citrate, a mixture of NaCitrate and NaCl). Hybridization may be carried out in solution or—more commonly—at least one component may be on a solid-phase support, e.g., nitrocellulose paper. Frequently used protocols employ a blocking reagent, such as casein from nonfat dried milk or bovine serum albumin, often in combination with denatured, fragmented salmon sperm DNA (or any other heterologous DNA of high complexity) and a detergent, such as SDS. Often a very high concentration of SDS is used as a blocking agent. Temperatures may be between 42 and 65° C. or higher, and buffers may be 3×SSC, 25 mM HEPES, pH 7.0, 0.25% SDS final.

Preferred is a method according to the present invention, wherein said portion of the said preselected guide RNA designed for binding to at least one target RNA specifically hybridizes with the target RNA with 15 or more nucleotides, preferably with 18 and more nucleotides, and more preferred with about 20 nucleotides or more. Preferred ranges are between 15 and 30 nucleotides, more preferred 18 to 25 nucleotides, and most preferred 20 to 24 nucleotides. Extensions to the hybridizing portions in the complex (3′ of the guide), are possible and preferred, and provide the advantage of a more stable formation the complex.

Further preferred is a method according to the present invention, wherein the target RNA comprises an rPAM (see above). In a preferred embodiment of the methods according to the present invention, the Cas nuclease can be modified in order to recognize a broader panel of rPAM sites, e.g., by replacing the key region in the interaction (PI) domain of Cas with the corresponding region in a panel of related Cas orthologs (see for example, for Cas9, Ma et al., Engineer chimeric Cas9 to expand PAM recognition based on evolutionary information. Nat Commun. 2019 Feb. 4; 10(1):560. doi: 10.1038/s41467-019-08395-8). This broadens the possible RNA targets in cells.

In the next step of the method according to the present invention the complex as formed between the least one CasΩ nuclease enzyme and the at least one preselected guide RNA as above is bound to the target RNA based on the sequence as designed for the preselected guide RNA as mentioned above. The CasΩ enzymes bind target nucleic acid independently of their ability to cleave target nucleic acid, and this flexibility is used in order to bind said at least one target RNA.

In the context of the present invention, a target RNA is any RNA of interest to be used as a trigger to cause cleaving and/or to be detected using the methods according to the present invention. Usually and preferably, the target RNA is a single-stranded RNA molecule, such as a messenger RNA, ribosomal RNAs, transfer RNAs, small RNAs, antisense RNAs, small nucleolar RNAs, microRNAs, piwiRNAs, long non-coding RNAs, spliced introns, and circular RNAs. The RNA can be of natural origin or artificially produced. The single stranded sensed RNA can be from a human cell, an animal cell, a plant cell, a cancerous cell, an infected cell, or a diseased cell, and/or can be derived from a virus, a parasite, a helminth, a fungus, a protozoan, a bacterium, or a pathogenic bacterium. The target RNA comprises a sequence that specifically hybridizes with a portion of the (non-naturally occurring) guide RNA as generated and used in the methods of the present invention.

Preferred is the complex according to the present invention, wherein said guide RNA comprises a sequence selected to be specific for a bacterium, a sequence selected to be specific for a virus, a sequence selected to be specific for a fungus, a sequence selected to be specific for a protozoan, a sequence selected to be specific for a genetic disorder, and a sequence selected to be specific for a proliferative disorder. Usually, said sequence is a complement or partial complement to the above target RNA.

In the last step of the method according to the present invention, the at least one CasΩ nuclease enzyme is cleaving (i.e., cutting, cleaving and/or nicking) nucleic acid molecules selected from dsDNA, ssDNA, and RNA. In contrast to the above mentioned “trigger” of specific RNA-binding to the target RNA, the nuclease-activity is non-specific. In contrast to other Cas-nucleases, like Cas13a (C2c2), that possesses an RNA-triggered non-specific RNase activity, and the type V effector protein, Cas12a, that possesses a dsDNA-triggered non-specific ssDNase activity, the present Cas-nuclease CasΩ possesses an RNA-triggered non-specific nuclease activity (see also Varble A, Marraffini L A. Three New Cs for CRISPR: Collateral, Communicate, Cooperate. Trends Genet. 2019; 35(6):446-456. doi:10.1016/j.tig.2019.03.009).

The methods according to the present invention can be performed in vivo or in vitro, for example in an organism, a cell, tissue, and/or part thereof, like a nucleus, or in an in vitro assay, like a diagnostic assay.

As mentioned above, in a second aspect thereof, the object of the present invention is solved by providing a complex comprising a CasΩ nuclease and at least one pre-selected guide RNA that, preferably specifically, is designed for binding to at least one target RNA. Preferred is the complex according to the present invention which further bound to a target RNA molecule having a sequence that is to at least 80%, preferably to more than 90%, more preferably to more than 95%, and most preferably 100% complementary to said guide RNA, and wherein said target RNA is preferably flanked by at least one rPAM.

The CasΩ nuclease can be selected from the above mentioned enzymes, as well as from fragments thereof that maintain the RNA-dependent nuclease activity as disclosed herein, i.e., fragments at least maintaining the RuvC domain. The polypeptide of the CasΩ nuclease can be provided depending on the use as intended, e.g., in vivo or in vitro, and is synthetically produced, generated by in vitro transcription, and/or cloned into a plasmid. The enzyme may be provided as purified or essentially purified isolated enzyme preparation. Complexes according to the present invention may also be prepared by a mixture of CasΩ nucleases, e.g., two or three or more nucleases or the fragments thereof as described.

Thus, the CasΩ polypeptide as used can be wild type CasΩ polypeptide, modified CasΩ polypeptide, or a fragment of a wild type or modified CasΩ polypeptide. The CasΩ polypeptide can be modified to increase nucleic acid binding affinity and/or specificity, alter an enzymatic activity, and/or change another property of the protein. For example, nuclease (i.e., DNase, RNase) domains of the CasΩ polypeptide can be modified, deleted, or inactivated. Alternatively, the CasΩ polypeptide can be truncated to remove domains that are not essential for the function of the protein, i.e., preferably the RNA-dependent nuclease activity.

Fusion proteins are provided herein comprising a CasΩ polypeptide, or a fragment or variant thereof, and an effector domain. The CasΩ polypeptide can be directed to a target site by a guide RNA, at which site the effector domain can modify or effect the targeted nucleic acid sequence. The effector domain can be a cleavage domain, an RNA modifying domain, a translational activation domain, a translational repressor domain, a processing/splicing factor, a domain affecting RNA localization, or a domain that recruits proteins affecting any of these functions. The fusion protein can further comprise at least one additional domain chosen from a nuclear localization signal, plastid signal peptide, mitochondrial signal peptide, signal peptide capable of protein trafficking to multiple subcellular locations, a cell-penetrating domain, or a marker domain, any of which can be located at the N-terminus, C-terminus, or an internal location of the fusion protein.

The CasΩ polypeptide can be located at the N-terminus, the C-terminus, or in an internal location of the fusion protein. The CasΩ polypeptide can be directly fused to the effector domain, or can be fused with a linker. In specific embodiments, the linker sequence fusing the CasΩ polypeptide with the effector domain can be at least 1, 2, 3, 4, 5, 6, 7, 8, 9, 10, 15, 20, 25, 30, 40, or 50 amino acids in length. For example, the linker can range from 1-5, 1-10, 1-20, 1-50, 2-3, 3-10, 3-20, 5-20, or 10-50 amino acids in length. The CasΩ polypeptide can also recruit the effector through a binding domain.

Preferred is the complex according to the present invention, wherein said nuclease comprises a nuclear localization signal. Said fusion nuclease comprising a nuclear localization signal and the complex as described herein as formed therewith are other embodiments of the present invention.

In some embodiments, the CasΩ polypeptide of the fusion protein can be derived from a wild type CasΩ protein. The CasΩ-derived protein can be a modified variant or a fragment. In some embodiments, the CasΩ polypeptide can be modified to contain a nuclease domain (e.g., a RuvC or RuvC-like domain) with reduced or eliminated nuclease activity. The nuclease domain can be modified by one or more deletion mutations, insertion mutations, and/or substitution mutations using known methods, such as site-directed mutagenesis, PCR-mediated mutagenesis, and total gene synthesis, as well as other methods known in the art.

The complex or complexes then comprise at least one pre-selected guide RNA that, preferably specifically, is designed for binding to at least one target RNA. How to design and choose the sequence(s) of the guide RNA generally depends on the sequence of the target RNA and the assay conditions; and how to design and choose such sequences is known to the person of skill in the art.

Preferred is the complex according to the present invention wherein said guide RNA molecule comprises a sequence that is to at least 80%, preferably to more than 90%, more preferably to more than 95%, and most preferably 100% complementary to a target RNA, and wherein said target RNA is preferably flanked by at least one rPAM. The guide RNA can be designed for binding to at least one target RNA, derived from naturally occurring sequences that are then modified in order to generate sequences in the molecule as desired. The guide RNA can further comprise additional modifications, such as labels or modified nucleotides, e.g., inosines, or the like. In case of both a naturally-occurring and/or a non-naturally occurring guide RNA, this can be produced in accordance with standard methods, e.g., synthetically produced, generated by in vitro transcription, and/or cloned into a plasmid or plasmids or other suitable vectors.

The parts of the nucleic acid molecules as used in the methods of the present invention that hybridize are complementary to at least 80%, preferably complementary to more than 90%, more preferably to more than 95%, and most preferably are 100% complementary to each other. Thus, the nucleotide sequence of said portion/part of said guide RNA that specifically hybridizes with the target RNA can be produced and/or modified in order to be complementary to at least 80%, preferably complementary to more than 90%, more preferably to more than 95%, and most preferably are 100% complementary to the target RNA.

Certain portions of the nucleic acid molecules as used in the methods according to the present invention are found and/or designed to specifically hybridize with complementary portions in other molecules. As known to the person of skill, for this, the hybridization and washing conditions are critical. If the sequences are 100% complementary, then a high stringency hybridization may be carried out. Nevertheless, according to the invention, the portions that hybridize and/or specifically hybridize are complementary to at least 80%, preferably complementary to more than 90%, more preferably to more than 95%, and most preferably are 100% complementary. The stringency of hybridization is determined by the hybridization temperature and the salt concentration in the hybridization buffer, and high temperature and low salt is more stringent. A commonly used washing solution is SSC (Saline Sodium Citrate, a mixture of NaCitrate and NaCl). Hybridization may be carried out in solution or—more commonly—at least one component may be on a solid-phase support, e.g., nitrocellulose paper. Frequently used protocols employ a blocking reagent, such as casein from nonfat dried milk or bovine serum albumin, often in combination with denatured, fragmented salmon sperm DNA (or any other heterologous DNA of high complexity) and a detergent, such as SDS. Often a very high concentration of SDS is used as a blocking agent. Temperatures may be between 42 and 65° C. or higher, and buffers may be 3×SSC, 25 mM HEPES, pH 7.0, 0.25% SDS final.

Preferred is a method according to the present invention, wherein said portion of the said preselected guide RNA designed for binding to at least one target RNA specifically hybridizes with the target RNA with 15 or more nucleotides, preferably with 18 and more nucleotides, and more preferred with about 20 nucleotides or more. Preferred ranges are between 15 and 30 nucleotides, more preferred 18 to 25 nucleotides, and most preferred 20 to 24 nucleotides. Extensions to the hybridizing portions in the complex (3′ of the guide), are possible and preferred, and provide the advantage of a more stable formation the complex.

The guide RNA can be further modified, preferably in order to introduce enhanced or new functionalities. For example, the 5′ and/or 3′ end of the guide RNA can be extended to be perfectly complementary to the target RNA, creating a dsRNA that can be edited with RNA-modifying enzymes (e.g., ADARs). The structure of the conserved CasΩ handle motif 5′ to the guide motif can be modified in order to stabilize the recognized hairpin structure or to promote binding by CasΩ. The 5′ and/or 3′ end of the guide RNA can be extended to further incorporate aptamer sequences. These aptamers can then recognize peptide or protein ligands fused to effector domains as used. Aptamers and their applications are well known in the art (see, for example, Rabiee N, Ahmadi S, Arab Z, Bagherzadeh M, Safarkhani M, Nasseri B, Rabiee M, Tahriri M, Webster T J, Tayebi L. Aptamer Hybrid Nanocomplexes as Targeting Components for Antibiotic/Gene Delivery Systems and Diagnostics: A Review. Int J Nanomedicine. 2020 Jun. 17; 15:4237-4256. doi: 10.2147/IJN.S248736. PMID: 32606675; PMCID: PMC7314593).

The complex according to the present invention is finally formed between the at least one CasΩ nuclease enzyme, and the at least one preselected guide RNA as above bound to the target RNA based on the sequence as designed for the preselected guide RNA as mentioned above. The CasΩ enzymes bind target nucleic acid independently of their ability to cleave target nucleic acid, and this flexibility is used in order to bind said at least one target RNA.

In the context of the present invention, a target RNA is any RNA of interest to be used as a trigger to cause cleaving and/or to be detected using the methods according to the present invention. Usually and preferably, the target RNA is a single-stranded RNA molecule, such as a messenger RNA, ribosomal RNAs, transfer RNAs, small RNAs, antisense RNAs, small nucleolar RNAs, microRNAs, piwiRNAs, long non-coding RNAs, spliced introns, and circular RNAs. The RNA can be of natural origin or artificially produced. The single stranded sensed RNA can be from a human cell, an animal cell, a plant cell, a cancerous cell, an infected cell, or a diseased cell, and/or can be derived from a virus, a parasite, a helminth, a fungus, a protozoan, a bacterium, or a pathogenic bacterium. As above, the target RNA comprises a sequence that specifically hybridizes with the guide portion of the (non-naturally occurring) guide RNA as generated and used in the methods of the present invention.

Another important aspect of the present invention is the diagnostic use of the complex and methods of the present invention. This aspect solves the object of the present invention by providing a method for detecting at least one target RNA in a cell, tissue, cellular nucleus, and/or sample, said method comprising: a) providing at least one ssDNA, dsDNA or RNA reporter nucleic acid in said cell, tissue, cellular nucleus, and/or sample, b) contacting said cell, tissue, cellular nucleus, and/or sample with at least one complex between at least one CasΩ nuclease enzyme and at least one preselected guide RNA, wherein said at least one preselected guide RNA comprises a guide sequence that is at least 90% complementary to the target RNA, and c) detecting, cleaving, cutting and/or nicking of said at least one ssDNA, dsDNA or RNA reporter nucleic acid, wherein detecting said cleaving the at least one reporter nucleic acid detects said at least one target RNA in said cell, tissue, cellular nucleus and/or sample.

As mentioned above, the parts of the nucleic acid molecules as used in the methods of the present invention that hybridize are complementary to at least 80%, preferably complementary to more than 90%, more preferably to more than 95%, and most preferably are 100% complementary to each other. Thus, the nucleotide sequence of said guide portion/part of said guide RNA that specifically hybridizes with the target RNA can be produced and/or modified in order to be complementary to at least 80%, preferably complementary to more than 90%, more preferably to more than 95%, and most preferably are 100% complementary to the target RNA.

When applying the respective complementarity and assay conditions, the present method can be used in order to detect mutations in the target RNA, but also RNAs that are undesired, present at a higher level in the cell or sample, and/or are foreign, such as, for example, from a human cell, an animal cell, a plant cell, a cancerous cell, an infected cell, or a diseased cell, and/or can be derived from a virus, a parasite, a helminth, a fungus, a protozoan, a bacterium, or a pathogenic bacterium.

In a preferred embodiment of the methods according to the present invention, said at least one target RNA is derived from a virus selected from Zika virus, human immunodeficiency virus (HIV), hepatitis B virus, hepatitis C virus, herpes virus, coronavirus, influenza, herpes simplex virus I, herpes simplex virus II, papillomavirus, rabies virus, cytomegalovirus, human serum parvo-like virus, respiratory syncytial virus, varicella-zoster virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, west Nile virus, coronavirus, yellow fever virus, and African swine fever virus.

In a preferred embodiment of the methods according to the present invention, said at least one target RNA is derived from a pathogenic bacterium selected from Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Treponema pallidum, Lyme disease spirochetes, Pseudomonas aeruginosa, Mycobacterium leprae, and Brucella abortus.

Preferred is the method according to the present invention, wherein the at least one target RNA is a mutated target RNA comprising at least one mutation compared to a control target RNA.

In a preferred embodiment of the methods according to the present invention, the at least one target RNA is derived from a gene the transcription and/or expression thereof is modified in response to external factors, such as, for example, metabolic factors or signals, hormones, pathogens, toxins, drugs, ageing and/or biotic or abiotic stress.

In a preferred embodiment of the methods according to the present invention, the target RNAs are selected to be environment, species, strain, disease, cell- and/or tissue specific. In this aspect, the methods of the invention help to identify and/or to group cells or organisms based on the target RNAs as selected. The at least one target RNA is preferably related to a condition selected from a viral infection, such as, for example, coronavirus infection, infection with a pathogen, a metabolic disease, cancer, neurodegenerative diseases, ageing, a drug, and biotic or abiotic stress.

In a further preferred embodiment of the methods according to the present invention, the at least one target RNA can be added to said cell, tissue and/or sample prior to step a), and/or wherein said method further comprises at least one step selected from in vitro transcription of DNA into RNA, reverse transcription of RNA into DNA, and—optimally—subsequent in vitro transcription of said DNA into RNA. This can be done in order to provide suitable or desired signal amplification. Usually, the target RNA in said cell, tissue or sample is present in a range of from about 500 fM to about 1 uM, e.g., from about 500 fM to about 1 nM, preferably is present in a range of from about 1 pM to about 1 nM. Optimally, the method can detect a single molecule per cell, tissue and/or sample.

Given the broad applicability of sequence-specific RNA recognition triggering DNA degradation, CasΩ nucleases provide several advantages. The COVID-19 pandemic has highlighted the need for inexpensive and rapid diagnostics that can detect even single-nucleotide differences. Even after the pandemic subsides, society will be more aware of the benefits of diagnostics and accepting of their use in everyday settings (e.g., the airport). CasΩ nucleases recognize a specific RNA target sequence, leading to degradation of, for example, a ssDNA or dsDNA or RNA reporter. The readout can be fluorescent (e.g., cleaving a reporter fused to a fluorophore and a quencher) or colorimetric (e.g., release of a nanoparticle as part of a lateral flow assay). The sequence-specificity of CasΩ nucleases can allow the diagnostic assay to differentiate even a single-nucleotide change, in a target RNA such as those associated with viral, in particular SARS-CoV-2, variants. Current CRISPR technologies based on Cas12a or Cas13 rely on recognition of dsDNA or ssRNA targets, triggering collateral cleavage of ssDNA or ssRNA reporters.

Smith C W, et al. (in: Probing CRISPR-Cas12a Nuclease Activity Using Double-Stranded DNA-Templated Fluorescent Substrates. Biochemistry. 2020 Apr. 21; 59(15):1474-1481. doi: 10.1021/acs.biochem.0c00140. Epub 2020 Apr. 7. PMID: 32233423; PMCID: PMC7384386) report a dsDNA substrate (probe-full) for probing Cas12a trans-cleavage activity upon target detection. A diverse set of Cas12a substrates with alternating dsDNA character were designed and studied using fluorescence spectroscopy. Smith et al. observed that probe-full without any nick displayed trans-cleavage performance that was better than that of the form that contains a nick. Different experimental conditions of salt concentration, target concentration, and mismatch tolerance were examined to evaluate the probe performance. The activity of Cas12a was programmed for a dsDNA frame copied from a tobacco curly shoot virus (TCSV) or hepatitis B virus (HepBV) genome by using crRNA against TCSV or HepBV, respectively. While on-target activity offered detection of as little as 10 pM dsDNA target, off-target activity was not observed even at 1 nM control DNAs. They demonstrated that trans-cleavage of Cas12a is not limited to ssDNA substrates, and Cas12a-based diagnostics can be extended to dsDNA substrates. However, this mode of detection still required a dsDNA target and therefore cannot detect an RNA target unless a reverse-transcription step is added beforehand.

There are other standard diagnostic technologies, such as PCR and LAMP. Another advantage of the present technologies is that they can be performed using lateral flow assays. The current technology can also provide single-nucleotide resolution normally associated with Cas nucleases, which are more difficult to achieve with PCR or LAMP.

While the basic principles of this aspect regarding the components as used are as above, in this aspect of the present invention, the detection of said at least one target RNA in said cell, tissue and/or sample relies on the detection of a cleaving of said at least one target nucleic acid in said sample by said nuclease enzyme, and detecting said cleaving of said at least one target nucleic acid thus detects said at least one target RNA in said cell, tissue and/or sample.

CasΩ can recognize RNA targets and degrade ssDNA and dsDNA. This allows the nuclease to directly sense RNAs without a reverse-transcription step, and it degrades inexpensive and stable ssDNA and dsDNA. Cas13, the leading technology, collaterally cleaves RNA. The associated RNA reporters are more expensive to synthesize and less stable than ssDNA or dsDNA, offering an immediate advantage for CasΩ. The ability to use dsDNA reporters further allows ways to boost the readout of cleavage activity, such as by creating dsDNA origami complexed with multiple fluorophores.

Examples for preferred in vitro diagnostic formats for methods of the present invention are lateral flow assays. Lateral flow assays are known to the person of skill, and operate on the same principles as enzyme-linked immunosorbent assays (ELISA). In essence, these tests run the liquid sample along the surface of a pad with reactive molecules that show a visual positive or negative result.

Preferred is therefore a method according to the present invention, wherein detecting said cleaving, cutting and/or nicking of the at least one reporter nucleic acid comprises detecting a change in the signal of a suitable label, such as a dye, a fluorophore, (e.g., detected by fluorescence detection or Raman spectroscopy), or electrical conductivity, and/or detecting the said cleaved at least one reporter nucleic acid fragment itself.

Another important aspect of the present invention is a method for modulating expression of at least one target RNA in a cell, tissue, cellular nucleus, and/or sample, wherein said at least one target RNA is selected from an mRNA, non-coding RNA and a viral RNA molecule, said method comprising: a) contacting said cell, tissue, cellular nucleus, and/or sample with b) at least one complex between at least one CasΩ nuclease enzyme and at least one preselected guide RNA, wherein said at least one preselected guide RNA comprises a sequence that is at least 90% complementary to the at least one target RNA, and c) binding the complex of b) to the at least one target RNA and thereby altering the stability, processing, localization, or translation of the at least one target RNA, whereby the binding in c) modulates the expression of at least one target RNA in the cell, tissue, cellular nucleus, and/or sample.

RNA targeting by CasΩ is used to impact translation of at least one target RNA in a cell, tissue, cellular nucleus, and/or sample, wherein said at least one target RNA is selected from an mRNA, non-coding RNA and a viral RNA molecule. This aspect of the present invention also allows multiplexable and sequence-specific gene silencing, which can be used for basic research, high-throughput screens, e.g., for anti-virals or other therapeutic substances. Targeting a target RNA of interest thus modulates gene expression in a sequence-specific manner, such as by altering mRNA stability, processing, or translation.

As mentioned above, the parts of the nucleic acid molecules as used in the methods of the present invention that hybridize are complementary to at least 80%, preferably complementary to more than 90%, more preferably to more than 95%, and most preferably are 100% complementary to each other. Thus, the nucleotide sequence of said portion/part of said guide RNA that specifically hybridizes with the target RNA can be produced and/or modified in order to be complementary to at least 80%, preferably complementary to more than 90%, more preferably to more than 95%, and most preferably are 100% complementary to the target RNA.

When applying the respective complementarity and assay conditions, the present method can be used in order to modulate the expression of at least one target RNA in a cell, tissue, cellular nucleus, and/or sample. Preferably, said at least one target RNA is selected from an RNA where a modulation of the expression will have a beneficial effect on the cell, tissue, cellular nucleus, and/or sample, such as an mRNA, non-coding RNA and a viral RNA molecule. In this method according to the present invention the cell, tissue, cellular cytoplasm, cellular nucleus, and/or sample is contacted with at least one complex between at least one CasΩ nuclease enzyme and at least one preselected guide RNA according to the present invention. Binding of the complex to the at least one target RNA will thereby alter the stability, processing, or translation of the at least one target RNA, and thus the binding of the complex modulates the expression of at least one target RNA in the cell, tissue, cellular nucleus, and/or sample. While the components and the conditions of the method are generally the same as described above, the complex as formed between the least one CasΩ nuclease enzyme and the at least one preselected guide RNA as above is bound to the target RNA based on the sequence as designed for the preselected guide RNA as mentioned above independently of the ability of CasΩ enzymes to cleave target nucleic acid, and this flexibility is used in order to bind said at least one target RNA. Thus, in this aspect, the CasΩ polypeptide can be modified to contain a nuclease domain (e.g., a RuvC or RuvC-like domain) with reduced or eliminated nuclease activity. The nuclease domain can be modified by one or more deletion mutations, insertion mutations, and/or substitution mutations using known methods, such as site-directed mutagenesis, PCR-mediated mutagenesis, and total gene synthesis, as well as other methods known in the art.

Preferably, fusion proteins are provided in this aspect, comprising a CasΩ polypeptide, or a fragment or variant thereof, and an effector domain. The CasΩ polypeptide can be directed to a target site by a guide RNA, at which site the effector domain can modify or effect the targeted nucleic acid sequence. The effector domain can be a cleavage domain, an RNA modifying domain, a translational activation domain, a translational repressor domain, a processing/splicing factor, a domain affecting RNA localization, or a domain that recruits proteins affecting any of these functions. The fusion protein can further comprise at least one additional domain chosen from a nuclear localization signal, plastid signal peptide, mitochondrial signal peptide, signal peptide capable of protein trafficking to multiple subcellular locations, a cell-penetrating domain, or a marker domain, any of which can be located at the N-terminus, C-terminus, or an internal location of the fusion protein. The CasΩ polypeptide can be located at the N-terminus, the C-terminus, or in an internal location of the fusion protein. The CasΩ polypeptide can be directly fused to the effector domain, or can be fused with a linker. In specific embodiments, the linker sequence fusing the CasΩ polypeptide with the effector domain can be at least 1, 2, 3, 4, 5, 6, 7, 8, 9, 10, 15, 20, 25, 30, 40, or 50 amino acids in length. For example, the linker can range from 1-5, 1-10, 1-20, 1-50, 2-3, 3-10, 3-20, 5-20, or 10-50 amino acids in length.

Preferred is the complex according to the present invention, wherein said nuclease comprises a nuclear localization signal (NLS), and respective signals are described herein and are known in the art. Said fusion nuclease comprising a nuclear localization signal and the complex as described herein as formed therewith are other embodiments of the present invention. Alternatively, no NLS can be included, which has the advantage of preventing collateral cleavage or degradation of nucleolar or mitochondrial DNA in eukaryotes.

Another important aspect of the present invention then relates to a method for editing the sequence of at least one target RNA in a cell, tissue, cellular nucleus, and/or sample, wherein said at least one target RNA is selected from an mRNA, non-coding RNA and a viral RNA molecule, said method comprising: a) contacting said cell, tissue, cellular nucleus, and/or sample with b) at least one complex between at least one modified and catalytically inactive CasΩ nuclease enzyme complexed with at least one RNA-modifying enzyme and at least one preselected guide RNA, wherein said at least one preselected guide RNA comprises a sequence that is at least 90% complementary to the at least one target RNA, and c) binding the complex of b) to the at least one target RNA, and editing of the at least one target RNA by said at least one RNA-modifying enzyme.

As mentioned above, the parts of the nucleic acid molecules as used in the methods of the present invention that hybridize are complementary to at least 80%, preferably complementary to more than 90%, more preferably to more than 95%, and most preferably are 100% complementary to each other. Thus, the nucleotide sequence of said portion/part of said guide RNA that specifically hybridizes with the target RNA and/or the rPAM can be produced and/or modified in order to be complementary to at least 80%, preferably complementary to more than 90%, more preferably to more than 95%, and most preferably are 100% complementary to the target RNA.

In this aspect of the invention, the complex is used to edit the sequence of at least one target RNA in a cell, tissue, cellular nucleus, and/or sample, wherein said at least one target RNA is selected from an mRNA, non-coding RNA and a viral RNA molecule. For example, different genetic diseases can be corrected by editing the RNA rather than the underlying DNA, offering a means to treat the disease without creating a permanent edit in the genome. There are a few editing approaches in the art, including modified Cas9 and Cas13 nucleases as well as oligonucleotides that recruit natural RNA-modifying enzymes (ADARs).

Preferably, fusion proteins are provided in this aspect, comprising a CasΩ polypeptide, or a fragment or variant thereof, and an effector domain. The CasΩ polypeptide can be directed to a target site by a guide RNA, at which site the effector domain can modify the targeted nucleic acid sequence. The fusion protein can further comprise at least one additional domain chosen from a nuclear localization signal, plastid signal peptide, mitochondrial signal peptide, signal peptide capable of protein trafficking to multiple subcellular locations, a cell-penetrating domain, or a marker domain, any of which can be located at the N-terminus, C-terminus, or an internal location of the fusion protein. The CasΩ polypeptide can be located at the N-terminus, the C-terminus, or in an internal location of the fusion protein. The CasΩ polypeptide can be directly fused to the effector domain, or can be fused with a linker. In specific embodiments, the linker sequence fusing the CasΩ polypeptide with the effector domain can be at least 1, 2, 3, 4, 5, 6, 7, 8, 9, 10, 15, 20, 25, 30, 40, or 50 amino acids in length. For example, the linker can range from 1-5, 1-10, 1-20, 1-50, 2-3, 3-10, 3-20, 5-20, or 10-50 amino acids in length. Preferred is a fusion of the catalytically inactivated version of CasΩ fused to an RNA-modifying enzyme (e.g., ADAR) that can direct targeted editing, resulting in an altered codon and different amino acid in the translated protein.

Binding of said at least one guide RNA-dependent CasΩ nuclease enzyme complex to said at least one target RNA can be detected by any suitable detection method as known to the person of skill, and may include chromatin immunoprecipitation (ChIP) methods using an antibody against the nuclease and RT-PCR primers for the RNA sequence of interest. The antibody is used to selectively precipitate the protein-RNA complex from the other RNA-protein complexes. The PCR primers allow specific amplification and detection of the target RNA sequence. Quantitative PCR (qPCR) technique allows the amount of target nucleic acid sequence to be quantified. The ChIP assay is amenable to array-based formats (ChIP-on-chip) or direct sequencing of the reverse transcribed DNA of the target RNA captured by the immunoprecipitated protein (ChIP-seq).

In a preferred embodiment of the methods according to the present invention, said at least one target RNA comprises a nucleic acid sequence that is specific for a disease state, such as, for example, for cells selected from the group consisting of cells exhibiting a genetic disorder, cells exhibiting a proliferative disorder, such as cancer cells, immune cells that produce autoantibodies, cells infected with bacterial or viral pathogens, bacterial pathogens, protozoan pathogens, cells of microbiota, and contaminating bacteria or archaea.

In another preferred embodiment of the methods according to the present invention, said at least one target RNA is single or initially double stranded. In the context of the present invention, a target RNA is any RNA of interest to be detected using the methods according to the present invention. Usually and preferably, the target RNA is a single-stranded RNA molecule, such as an mRNA, viral RNA or non-coding RNAs. The RNA can be of natural origin or artificially produced. The single stranded target RNA can be from a human cell, an animal cell, a plant cell, an immune cell, a cancerous cell, an infected cell, or a diseased cell, and/or can be derived from a virus, a parasite, a helminth, a fungus, a protozoan, a bacterium, or a pathogenic bacterium.

In a preferred embodiment of the methods according to the present invention, said method is performed in vivo, for example in a cell, tissue or in a bacterium, fungus, plant or animal, or in vitro, in a sample. The sample can be a solid or liquid sample, and can be selected from a sample comprising cells, and an acellular in vitro sample. The cells are preferably plant cells or animal cells, such as mammalian cells, preferably human cells. The sample can be a biological sample, preferably obtained from a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. In one aspect, the cells, tissues and/or samples can be crude samples and/or wherein the one or more nucleic acid molecules are not purified or amplified from the sample prior to application of the methods. In another aspect, the cells, tissues and/or samples can be purified or partially purified (enriched) samples and/or the one or more nucleic acid molecules are purified or amplified from the sample prior to application of the methods. In another aspect, the cells can be part of an environmental sample such as air, a natural body of water (e.g., river, lake, ocean), wastewater, or soil.

The methods according to the present invention can be partially or fully automated, e.g., performed fully or in part by robots. The methods according to the present invention can involve the use of computers and respective databases for performing and/or analysis of the results as obtained.

In a preferred embodiment of the methods according to the present invention, more than one guide RNAs are selected, designed, generated (produced, see above) and used, each specifically hybridizing with a different portion of said target RNA in one or more samples, tissues and/or cells, and preferably in several or even a multitude of samples, tissues and/or cells.

In the context of altering gene expression, RNA editing, or programmable viral or cell removal according to the present invention, the present system could, for example, target anywhere from 2 to 7 genetic loci by cloning multiple guide RNAs into a single plasmid. The guide RNAs can be expressed individually from separate promoters or combined into a CRISPR array transcribed from a single promoter. These multiplex guide RNA vectors can conceivably be suitably combined with the aforementioned CasΩ nucleases in order be used in the aspects of the invention.

In another aspect of the methods according to the present invention, said methods, at least in part, comprise a quantitative analysis. Preferred is therefore a step comprising detecting the amount of the nucleic acid as cleaved in said sample, tissue and/or cell. Preferably the amount per sample, tissue and/or cell is determined, when compared to a control. Quantification assays are known to the person of skill, and may include absorbance (e.g., UV, spectrophotometry) and/or fluorescence assays, and real-time PCR. The assays can quantify the amount(s) and/or ratios of the nucleic acid(s) as quantified as a single value (e.g., as the result or at the “end” of the assay as used) or can monitor changes over time in the nucleic acids, i.e., preferably further comprising detecting a change in the amount of said cleaved nucleic acid(s), in particular when compared to a control.

In yet another aspect of the methods according to the present invention, multiple labels and/or markers are used. Markers can be used both for the nucleic acid molecules that form part of the assays, as well as the protein components (e.g., nuclease and/or fusions). Labels and markers can be included into the components of the assays (in particular the nucleic acids and/or the proteins), as well as constitute moieties that are attached, either covalently or non-covalently.

Another aspect of the present invention then relates to a method for detecting a medical condition in a cell, tissue or organism, such as a mammal, preferably a human, wherein said condition is related to the presence of, expression of and/or mutation(s) in at least one target RNA. The method comprises performing the method according to the invention as above, and detecting said medical condition based on the nucleic acids cleavage as caused by the presence of, expression of and/or mutation(s) in said at least one target RNA as detected.

The medical conditions that can be detected using the present invention are those that are related to the at least one target RNA molecule. As explained above, the target RNA can either itself constitute the origin of the condition or disease, for example in case of infections, e.g., viral, such as, for example, coronavirus infection, bacterial and/or fungal infections of the cells, tissues and/or samples as tested. Other conditions may relate more indirectly to the at least one target RNA molecule, for example in case of an RNA that is aberrantly transcribed (present or found), expressed, processed (e.g., splicing) and/or mutated. The target RNA molecule can be present in an increased or decreased amount when compared to a healthy control (e.g., a control based on a group of healthy or diseased samples).

Examples for preferred in vitro diagnostic formats for methods of the present invention are lateral flow assays. Lateral flow assays are known to the person of skill and operate on the same principles as enzyme-linked immunosorbent assays (ELISA). In essence, these tests run the liquid sample along the surface of a pad with reactive molecules that show a visual positive or negative result.

As mentioned above, in a preferred embodiment of the methods according to the present invention, the at least one target RNA can be added to said cell, tissue and/or sample prior to step a), and/or wherein said method further comprises at least one step selected from in vitro transcription of DNA into RNA, reverse transcription of RNA into DNA, and—optimally—subsequent in vitro transcription of said DNA into RNA. This can be done in order to provide suitable or desired signal amplification. Usually, the target RNA in said cell, tissue or sample is present in a range of from about 500 fM to about 1 uM, e.g., from about 500 fM to about 1 nM, preferably is present in a range of from about 1 pM to about 1 nM. Optimally, the method can detect a single molecule per cell, tissue and/or sample.

Another important aspect of the present invention then relates to the use of the present invention in medicine.

One aspect of the present invention is a method for specifically inactivating an undesired cell or virus, comprising contacting said cell or virus with a complex according to the present invention as described herein, wherein the guide RNA, particularly the sequence thereof, is specifically selected/designed for said undesired cell or virus to be inactivated or non-edited cell as described herein.

Another aspect of the present invention is the complex according to the present invention as described herein, for use in the prevention and/or treatment of diseases, such as for example, for use in the prevention and/or treatment of infections and/or genetic disorder, such as proliferative disorders, such as cancer, fungal, protozoan, bacterial and/or viral infections.

Embodiments are clearing an infecting DNA or RNA virus from a cell and (specific) killing of an undesired cell because it contains a cancer mutation.

Yet another aspect of the present invention is a method for preventing and/or treating a disease, such as for example, an infection and/or genetic disorder, such as a proliferative disorder, such as cancer, fungal, protozoan, bacterial and/or viral infections, an autoimmune disease, comprising administering to a subject in need of such treatment an effective amount of the complex according to the present invention.

The present invention can be used for sequence-specific cell killing. There are numerous applications where killing a cell in a sequence-specific manner is desirable. Specific examples are selective killing of cancer cells, immune cells that produce autoantibodies, cells infected with bacterial or viral pathogens, bacterial pathogens, or contaminating bacteria or archaea in an industrial culture. Target RNA recognition in the nucleus (e.g., using CasΩ fused to a nuclear localization signal in eukaryotes) leads to extensive dsDNA cleavage, and killing of the cell. If the target RNA is absent in the cell or sample, or contains mutations, then the cell is spared. This effect particularly applies to cancer cell, bacteria and archaea. There is no available approach for programmable and sequence-specific killing in eukaryotes; instead, the field has focused on improvements that boost the editing efficiency.

CasΩ thus offers the first means to achieve sequence-specific killing/inactivation of prokaryotic and eukaryotic cells. In eukaryotes this could offer a unique means to kill cancer cells based on their unique mutations, as well as specific immune cells based on their differentiated genetic material encoding specific antibodies. This approach leads to new therapies, e.g., for treating specific autoimmune disorders, and can become a standard approach for enriching edited cells in a population.

The inventive approach is also used to fight infectious diseases. Delivery of CasΩ to eukaryotic cells infected with virus or bacteria aids the immune system by recognizing the viral or bacterial RNA and then destroying the virus or bacterial DNA. These treatments result in death of the infected host cell, which stops disease spread and activates the immune system further. Because CasΩ is selective, delivery to non-infected cells not containing the complementary viral or bacterial RNA causes an inert response.

The inventive approach is also used to treat/regulate/engineer microbial populations important for industry and medicine, for example, if a certain strain of bacteria within a mammalian, e.g., human, microflora is correlated with obesity, it is targeted for cell death without killing other in situ bacterial populations. Such treatments will also be able to provide avenues to combat antibiotic resistant strains of bacteria.

In this aspect, the complex or a nucleic acid encoding all or parts of it is used as the actual active ingredient in the prevention and/or treatment. Delivery of the complex to a patient a cell or a sample can be done in any suitable way, for example as a pharmaceutical composition comprising the isolated components of at least one complex (polypeptide and/or nucleic acid) according to the present invention together with suitable stabilizers or carriers. Another embodiment is the provision of the complex encoded on at least one nucleic acid vector to the patient, cell, tissue, sample or nucleus. These pharmaceutical compositions and uses thereof constitute preferred embodiments of the present invention. This aspect also includes the step of monitoring a treatment.

Another aspect is the use of the complex and the method of the present invention for the counterselection of non-edited cells to improve overall gene editing outcomes, as described above. This approach can select for any desired edits as introduced that disrupt the target RNA sequence, the rPAM, its accessibility, and/or its transcription.

Yet another aspect of the present invention then relates to a method for treating a disease or medical condition in a cell, tissue or organism, such as a mammal, preferably a human, wherein said condition is related to the presence of, expression of and/or mutation(s) in at least one target RNA.

This aspect combines the diagnostic approach of the invention with a separate “regular” medical treatment, and also includes the use for monitoring the treatment. The method comprises providing a suitable treatment to said cell, tissue or organism, in particular a specific medical treatment, performing the method according to the invention as above, and modifying said treatment of said disease or medical condition based on the presence of, expression of and/or mutation(s) in said at least one target RNA as detected. The medical conditions that can be detected using the present invention are those that are related to the at least one target RNA molecule. As explained above, the target RNA can either itself constitute the origin of the condition or disease, for example in case of infections, e.g., viral, such as, for example, coronavirus infection, bacterial and/or fungal infections of the cells, tissues and/or samples as tested. Other conditions may relate more indirectly to the at least one target RNA molecule, for example in case of an RNA that is aberrantly transcribed (present or found), expressed, processed and/or mutated. The target RNA molecule can be present in an increased or decreased amount when compared to a healthy control (e.g., a control based on a group of healthy or diseased samples).

In a preferred embodiment of the methods for treatment according to the present invention, said at least one target RNA is single or initially double stranded. The single stranded target RNA can be/relates to from a human cell, an animal cell, a plant cell, an immune cell, a cancerous cell, an infected cell, or a diseased cell, and/or can be derived from a virus (see above), a parasite, a helminth, a fungus, a protozoan, a bacterium, or a pathogenic bacterium (see above). In a preferred embodiment of the methods according to the present invention, the at least one target RNA is derived from/related to a gene the transcription and/or expression thereof is modified in response to external factors, such as, for example, metabolic factors or signals, hormones, pathogens, toxins, drugs, ageing and/or biotic or abiotic stress. Consequently, the at least one target RNA is preferably related to a condition selected from a viral infection, such as, for example, coronavirus infection, infection with a pathogen, a metabolic disease, cancer, neurodegenerative diseases, ageing, a drug, and biotic or abiotic stress). Usually, the presence of or the increased or decreased amount of the target RNA is indicative for the presence of said disease or condition. Another aspect of the method relates to a monitoring of the amount or presence of said target RNA during a treatment of said individual, patient or organism, in particular the individual, patient or organism from which the cell, tissue and/or sample has been obtained. The attending physician will then adjust the treatment accordingly, i.e., provide more with antiviral chemotherapeutics and/or biologics, if required. This treatment schedule can be repeated, if required.

Finally, again similarly as above, designed guide RNAs (e.g., specific for a bacterial or fungal target nucleic acid) could be used with a sample containing an infecting bacterial or fungal pathogen (e.g., Clostridioides difficile in a stool sample, Pseudomonas aeruginosa in a sputum sample) to identify specific markers of antibiotic resistance to determine the best regime of antibiotics to administer to a patient.

In another preferred aspect thereof, the object of the present invention is solved by providing a detection system for a target RNA comprising a) at least one preselected guide RNA designed to bind to at least one portion of said target RNA, wherein said at least one preselected guide RNA comprises a sequence that is at least 90% complementary to the target RNA, and b) at least one CasΩ nuclease enzyme. Preferred is a detection system for the parallel detection of several target RNAs in parallel, comprising a set of several guide RNAs for said several target RNAs. Another detection system comprises several guide RNAs that hybridize at several positions on one target RNA.

The parts of the nucleic acid molecules as used in the methods of the present invention that hybridize are complementary to at least 80%, preferably complementary to more than 90%, more preferably to more than 95%, and most preferably are 100% complementary to each other. Thus, the nucleotide sequence of said portion/part of said guide RNA that specifically hybridizes with the target RNA can be produced and/or modified in order to be complementary to at least 80%, preferably complementary to more than 90%, more preferably to more than 95%, and most preferably are 100% complementary to the target RNA.

This aspect of the present invention provides the components, e.g., preselected guide RNA nucleic acid molecule and at least one CasΩ nuclease enzyme as above for performing the methods according to the invention as a detection system, for example as a part of a diagnostic kit. The system may also be used in a therapeutic kit, or a pharmaceutical composition comprising the isolated components of at least one complex (polypeptide and/or nucleic acid) according to the present invention together with suitable stabilizers or carriers. Another embodiment is the provision of the complex encoded on at least one nucleic acid vector to the patient, cell, tissue, sample or nucleus.

Preferably, said system is provided in one or more containers, and comprises suitable enzymes, buffers, and excipients, as well as instructions for use. The components can be—at least in part—immobilized on a substrate, wherein said substrate can be exposed to said cell, tissue and/or sample. The detection system can be applied to multiple discrete locations on said substrate, such as a flexible materials substrate, for example a chip. The flexible materials substrate can be a paper substrate, a fabric substrate, or a flexible polymer-based substrate.

Yet another aspect of the present invention the relates to the use of the complex according to the present invention as described herein for cleaving a nucleic acid molecule selected from dsDNA, ssDNA, and RNA, for detecting at least one target RNA in a cell, tissue, cellular nucleus, and/or sample, for modulating expression of at least one target RNA in a cell, tissue, cellular nucleus, and/or sample, for editing the sequence of at least one target RNA in a cell, tissue, cellular nucleus, and/or sample, for specifically inactivating an undesired cell or virus, or for decontaminating a preparation from an undesired contaminant, in accordance with the methods as disclosed herein. Preferably, the object of the present invention is solved by providing the use of the CasΩ/guide RNA nucleic acid complex for performing a method according to any one of the aspects as above, in particular for detecting a target RNA, a viral target RNA, a target RNA transcribed from a disease marker, the treatment of diseases, and/or for generating an expression profile for one or more target RNAs, as mentioned above.

The embodiments described herein can be used for wide-ranging applications, such as diagnosing medical conditions to inform the course of treatment, identifying SNPs associated with health outcomes or disease, such as in acute sepsis, determining the identity of pathogen, virulence factors, resistance markers, SNPs, viral detection, i.e., the identity, and/or viral variants (see, for example, SARS CoV-2 as disclosed herein), cancer diagnostics, in cancer samples, such as biopsies, determining mutations, and/or SNPs, identifying microbial contaminants in potable water, identifying viral or microbial contaminants in fermentations or cell cultures, identifying plant or insect variants, or identifying key microbial members in mixed communities (e.g., in the gut, soil, water), such as analysis of microbiomes and/or microbial sentinels (i.e., commensal bacteria that serve as reporters for non-invasive measurements), in particular the identity, relative abundance, resistance markers, metabolic genes, phylum/genus/species/strain-specific genes, and the like, and the tracking of viral or bacterial spread in vivo, such as in whole organisms, or based on samples, for example taken from the environment (e.g., spread of viruses or resistant bacteria detected in waste water samples, etc.).

In the context of the present invention, unless explicitly mentioned otherwise, the term “about” shall mean a value as given +/−10%.

Cas12 nucleases (within Type V CRISPR-Cas systems) are known to recognize dsDNA, thereby eliciting cleavage of the bound dsDNA and subsequent degradation of ssDNA. Cas12a is the representative example. Given its similarity to the established Cas nuclease Cas12a, CasΩ was presumed to target DNA. The only exception within Cas12 nucleases is Cas12g, which recognizes RNA and degrades RNA and ssDNA. CasΩ as used here was originally classified as Cas12a based on its similarity, although it has distinct domains and sometimes appears with Cas12a.

As stated herein, the present invention particularly relates to the following items.
Item 1. A complex comprising a CasΩ nuclease and at least one preselected guide RNA designed for binding to at least one target RNA.
Item 2. The complex according to claim 1, further bound to a target RNA molecule having a sequence that is at least 90% complementary to said guide RNA, and wherein said target RNA is preferably flanked by at least one rPAM.
Item 3. The complex according to Item 1 or 2, wherein said guide RNA comprises a sequence selected to be specific for a bacterium, a sequence selected to be specific for a virus, a sequence selected to be specific for a fungus, a sequence selected to be specific for a protozoan, a sequence selected to be specific for a genetic disorder, and a sequence selected to be specific for a proliferative disorder.
Item 4. The complex according to any one of Items 1 to 3, wherein said nuclease comprises a nuclear localization signal.
Item 5. Method for cleaving a nucleic acid molecule selected from dsDNA, ssDNA, and RNA, comprising the steps of a) providing at least one CasΩ nuclease enzyme, b) providing at least one preselected guide RNA, c) forming a complex between the least one CasΩ nuclease enzyme and the at least one preselected guide RNA, d) binding of the complex of c) to a target RNA based on the at least one preselected guide RNA, and e) cleaving said nucleic acid molecule selected from dsDNA, ssDNA, and RNA by the at least one CasΩ nuclease enzyme.
Item 6. A method for detecting at least one target RNA in a cell, tissue, cellular nucleus, and/or sample, said method comprising: a) providing at least one ssDNA, dsDNA or RNA reporter nucleic acid in said cell, tissue, cellular nucleus, and/or sample, b) contacting said cell, tissue, cellular nucleus, and/or sample with at least one complex between at least one CasΩ nuclease enzyme and at least one preselected guide RNA, wherein said at least one preselected guide RNA comprises a sequence that is at least 90% complementary to the target RNA, and c) detecting a cleaving, cutting and/or nicking of said at least one ssDNA, dsDNA or RNA reporter nucleic acid, wherein detecting said cleaving the at least one reporter nucleic acid detects said at least one target RNA in said cell, tissue, cellular nucleus and/or sample.
Item 7. The method according to Item 6, wherein detecting said cleaving, cutting and/or nicking of the at least one reporter nucleic acid comprises detecting a change in the signal of a suitable label, such as a dye, a fluorophore, or electrical conductivity, and/or detecting the said cleaved at least one reporter nucleic acid fragment itself.
Item 8. The method according to Item 6 or 7 wherein the at least one target RNA is a mutated target RNA comprising at least one mutation compared to a control target RNA.
Item 9. A method for modulating expression of at least one target RNA in a cell, tissue, cellular nucleus, and/or sample, wherein said at least one target RNA is selected from an mRNA, non-coding RNA and a viral RNA molecule, said method comprising: a) contacting said cell, tissue, cellular nucleus, and/or sample with at least one complex between at least one CasΩ nuclease enzyme and at least one preselected guide RNA, wherein said at least one preselected guide RNA comprises a sequence that is at least 90% complementary to the at least one target RNA, and c) binding the complex of b) to the at least one target RNA and thereby altering the stability, processing, localization, or translation of the at least one target RNA, whereby the binding in c) modulates the expression of at least one target RNA in the cell, tissue, cellular nucleus, and/or sample.
Item 10. A method for editing the sequence of at least one target RNA in a cell, tissue, cellular nucleus, and/or sample, wherein said at least one target RNA is selected from an mRNA, non-coding RNA and a viral RNA molecule, said method comprising: a) contacting said cell, tissue, cellular nucleus, and/or sample with at least one complex between at least one modified and catalytically inactive CasΩ nuclease enzyme complexed with at least one RNA-modifying enzyme and at least one preselected guide RNA, wherein said at least one preselected guide RNA comprises a sequence that is at least 90% complementary to the at least one target RNA, and c) binding the complex of b) to the at least one target RNA, and editing of the at least one target RNA by said at least one RNA-modifying enzyme.
Item 11. The method according to any one of Items 5 to 10, wherein the at least one target RNA comprises a nucleic acid sequence that is specific for a disease state, such as, for example, for cells selected from the group consisting of cells exhibiting a genetic disorder, cells exhibiting a proliferative disorder, such as cancer cells, immune cells that produce autoantibodies, cells infected with bacterial or viral pathogens, bacterial pathogens, protozoan pathogens, cells of microbiota, and contaminating bacteria or archaea.
Item 12. The complex according to Item 3 or 4 for use in the prevention and/or treatment of diseases, such as for example, for use in the prevention and/or treatment of infections and/or genetic disorder, such as proliferative disorders, such as cancer, fungal, protozoan, bacterial and/or viral infections.
Item 13. Method for specifically inactivating an undesired cell or virus, comprising contacting said cell or virus with a complex according to any one of Items 1 to 4, wherein said guide RNA is specifically selected for said undesired cell or virus to be inactivated.
Item 14. A method for preventing and/or treating a disease, such as for example, an infection and/or genetic disorder, such as a proliferative disorder, such as cancer, fungal, protozoan, bacterial and/or viral infections, an autoimmune disease, comprising administering to a subject in need of such treatment an effective amount of the complex according to Items 3 or 4.
Item 15. Use of the complex according to any one of Items 1 to 4 for cleaving a nucleic acid molecule selected from dsDNA, ssDNA, and RNA, for detecting at least one target RNA in a cell, tissue, cellular nucleus, and/or sample, for modulating expression of at least one target RNA in a cell, tissue, cellular nucleus, and/or sample, for editing the sequence of at least one target RNA in a cell, tissue, cellular nucleus, and/or sample, for specifically inactivating an undesired cell or virus, for decontaminating a preparation from an undesired contaminant, or for removing cells that remain unedited by a method according to Item 10.
Item 16. The complex or the method according to any one of Items 1 to 13, wherein said guide RNA molecule comprises a sequence that is to at least 80%, preferably to more than 90%, more preferably to more than 95%, and most preferably 100% complementary to a target RNA.
Item 17. Method for specifically removing, inactivating and/or killing undesired cells that have not been edited by a method according to Item 10, comprising contacting the cells with a complex according to any one of Items 1 to 4, wherein said guide RNA is specifically selected for said unedited cell to be removed, inactivated and/or killed.

The present invention will now be further described in the following examples with reference to the accompanying Figures, nevertheless, without wanting to be limited thereto. For the purposes of the present invention, all references as cited herein are incorporated by reference in their entireties. The present disclosure includes as sequence listing comprising SEQ ID Nos: 1 to 67 as part of the description, which is also incorporated by reference in its entirety.

FIG. 1 shows that CasΩ forms three distinct clades among Class 2 Type V CRISPR-Cas nucleases. Maximum likelihood phylogeny of Class 2 Type V CRISPR-Cas protein sequences was generated, including three distinct monophyletic CasΩ clades, as typified by the representative nucleases SmCasΩ, SuCasΩ, and ca40CasΩ. CasΩ nucleases do not share the last common ancestor with Cas12a.

FIG. 2 shows amino acid conservation between the RuvC-I and RuvC-III motifs in CRISPR-SuCasΩ nucleases. Nuclease orthologues from the SuCasΩ phylogenetic clade exhibit unique amino acid composition, e.g., between the RuvC-I and RuvC-II catalytic motifs, comprising an insertion of multiple conserved amino acid motifs compared to non-CasΩ nucleases, such as Cas12a. Furthermore, SuCasΩ orthologues exhibit unique amino acid composition between the RuvC-II and RuvC-III catalytic motifs, comprising a deletion of amino acids compared to non-CasΩ nucleases, such as Cas12a. Relative entropy is shown in bits. High entropy indicates high certainty that a given amino acid is present in an orthologous motif based on the alignment of 16 SuCasΩ orthologues.

FIG. 3 shows amino acid conservation between the RuvC-I and RuvC-III motifs in CRISPR-SmCasΩ nucleases. Nuclease orthologues from the SmCasΩ phylogenetic clade exhibit unique amino acid composition, e.g., between the RuvC-I and RuvC-II catalytic motifs, comprising an insertion of multiple conserved amino acid motifs compared to non-CasΩ nucleases, such as Cas12a. Furthermore, SmCasΩ orthologues exhibit unique amino acid composition between the RuvC-II and RuvC-III catalytic motifs, comprising a deletion of amino acids compared to non-CasΩ nucleases, such as Cas12a. Relative entropy is shown in bits. High entropy indicates high certainty that a given amino acid is present in an orthologous motif based on the alignment of 36 SmCasΩ orthologues.

FIG. 4 shows amino acid conservation between the RuvC-I and RuvC-III motifs in CRISPR-ca40CasΩ nucleases. Nuclease orthologues from the ca40CasΩ phylogenetic clade exhibit unique amino acid composition, e.g., between the RuvC-I and RuvC-II catalytic motifs, comprising an insertion of multiple conserved amino acid motifs compared to non-CasΩ nucleases, such as Cas12a. Furthermore, ca40CasΩ orthologues exhibit unique amino acid composition between the RuvC-II and RuvC-III catalytic motifs, comprising a deletion of amino acids compared to non-CasΩ nucleases, such as Cas12a. Relative entropy is shown in bits. High entropy indicates high certainty that a given amino acid is present in an orthologous motif based on the alignment of 15 ca40CasΩ orthologues.

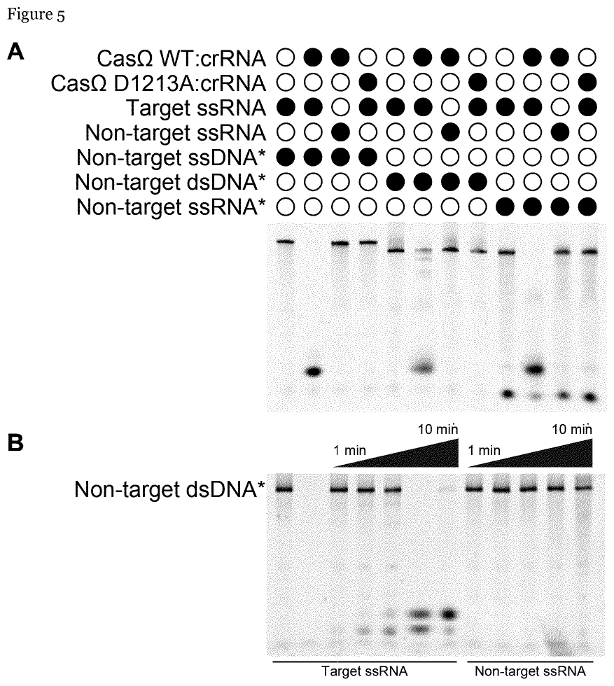

FIG. 5 shows that CasΩ recognizes RNA and cleaves RNA, ssDNA, and dsDNA in vitro. Purified SuCasΩ and a designed guide RNA (crRNA) were combined with an unlabeled target or non-target RNA as well as labeled non-target single stranded DNA (ssDNA), double-stranded DNA (dsDNA), and single stranded RNA (ssRNA). (A) Only in the presence of the RNA target did SuCasΩ degrade the non-target ssDNA, dsDNA, and ssRNA. (B) In the presence of non-target RNA, SuCasΩ did not degrade the ssDNA, dsDNA, and ssRNA. This activity (in particular, RNA target recognition and dsDNA collateral degradation) represents an entirely unique activity for CRISPR nucleases.

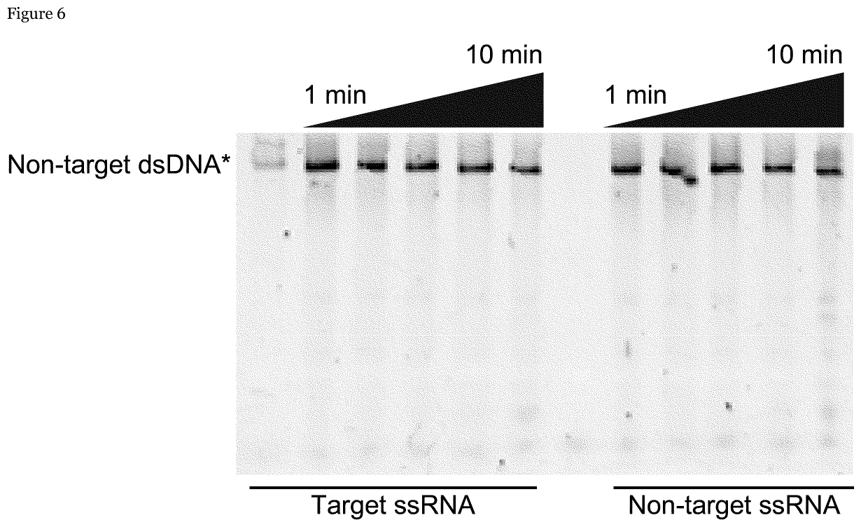

FIG. 6 shows that the RNA-triggered DNA degradation by CasΩ in vitro depends on the RuvC domain. SuCasΩ was mutated in two sites within the RuvC motifs associated with DNA cleavage. The cleavage assays were conducted as described in the previous figure. In this case, mutating the RuvC domain eliminated the RNA-triggered degradation of dsDNA.

FIG. 7 shows that CasΩ degrades ssDNA following RNA target recognition in vitro. RNA-triggered SuCasΩ activity was tested in vitro with ssDNA. The ssDNA was labeled with a fluorophore for fluorescence detection. The results show that ssDNA is also degraded by triggered SuCasΩ. Target ssDNA and dsDNA did not trigger SuCasΩ activity.

FIG. 8 shows that CasΩ degrades plasmid DNA following RNA target recognition in vitro. RNA-triggered SuCasΩ activity was tested in vitro with plasmid DNA. The plasmid was detected by running the nucleic acid products on an agarose gel and staining with ethidium bromide. The results show that plasmid DNA is also degraded by triggered SuCasΩ.

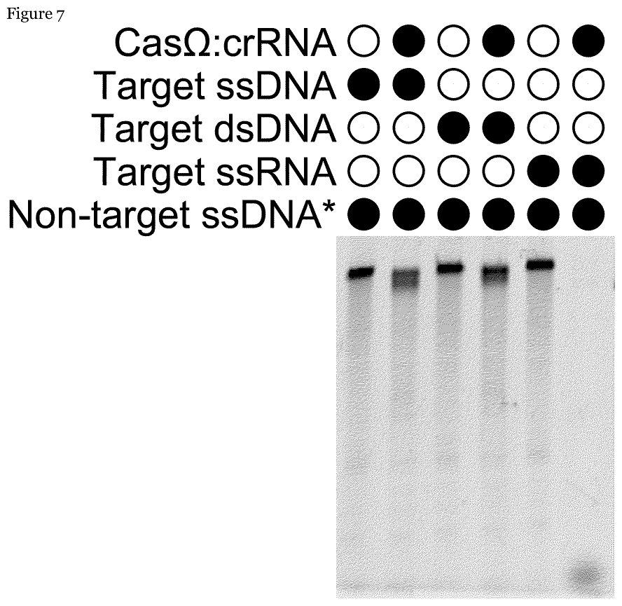

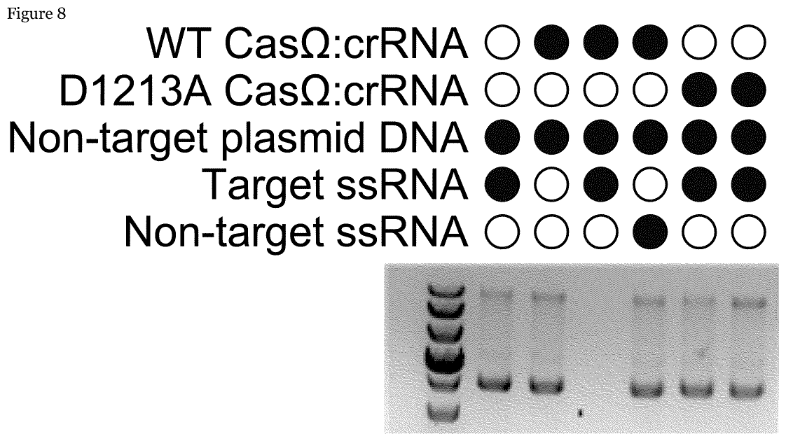

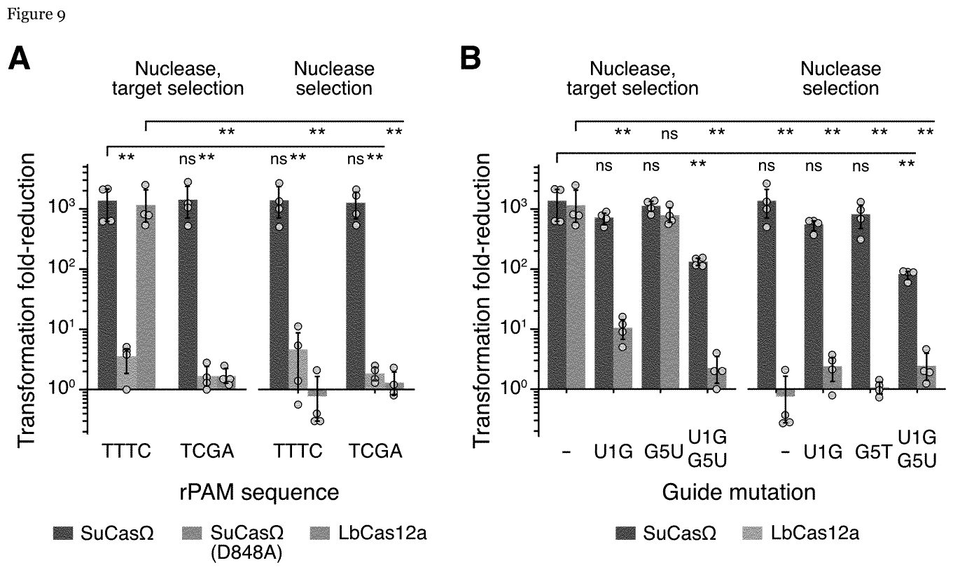

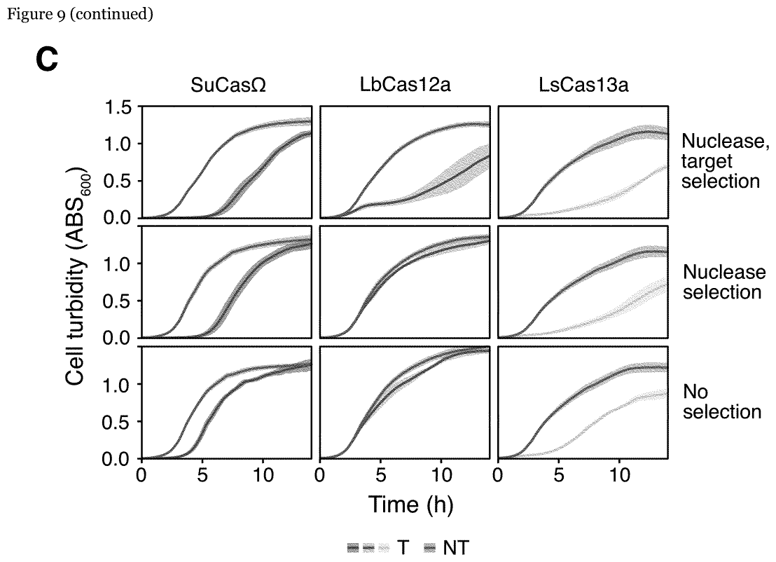

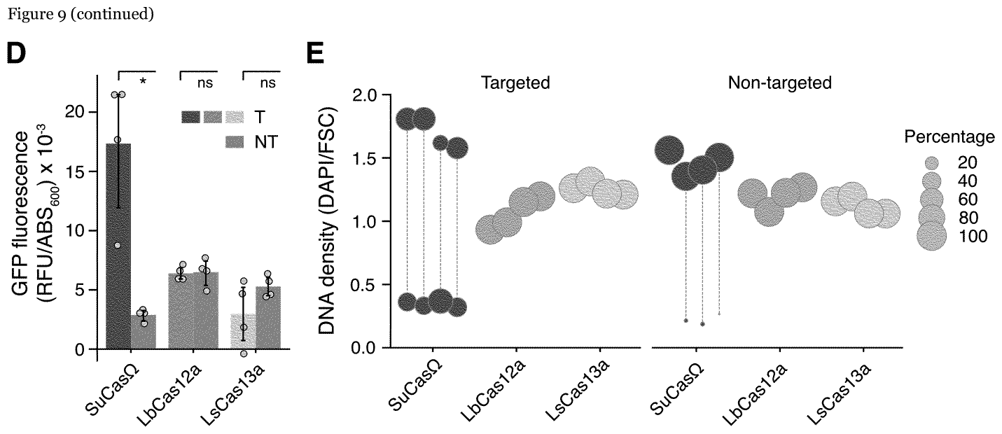

FIG. 9 shows that CasΩ impairs growth following target recognition in E. coli. The activity of SuCasΩ was assessed without selecting for the target plasmid or any plasmids. (A-B) Transformation-fold reduction when transforming the SuCasΩ plasmid into cells already harboring the crRNA plasmid and a target/non-target plasmid. Different PAMs ans rPAMs and target mismatches were tested with or without selection for the target plasmid. The rPAMs are reported as the DNA reverse complement to correspond with the PAM for Cas12a (e.g., the 5′-GAAA-3′ rPAM is reported as 5′-TTTC-3′. SuCasΩ but not Cas12a reduced plasmid transformation even when not selecting for the target plasmid. (C) Assessing growth of E. coli cells expressing different nucleases under different selection conditions. SuCasΩ and LsCas13a but not LbCas12a reduced growth even in the absence of a selecting antibiotic. LsCas13a is known to collaterally degrade cellular RNAs upon target recognition, yielding a similar effect on growth. It is furthermore shown that CasΩ targeting elicits an SOS response, cytotoxicity, and loss of DNA in E. coli. The impact of targeting by SuCasΩ compared to other nucleases was further assessed in E. coli. (D) Measuring the SOS response using the recA promoter driving GFP expression. GFP fluorescence was measured after 4-h induction of the nuclease and guide RNA, all in the absence of a selecting antibiotic. Only SuCasΩ significantly elicited the SOS response compared to a non-target control. (E) Assessing cell morphology and DNA content. Cells were stained with the DNA binding dye DAPI and assessed by flow cytometry analysis. Only cells with SuCasΩ targeting led to a bifurcation in the population, with some cells becoming filamentous while others becoming small and containing less DNA. Both reflect extensive DNA damage.

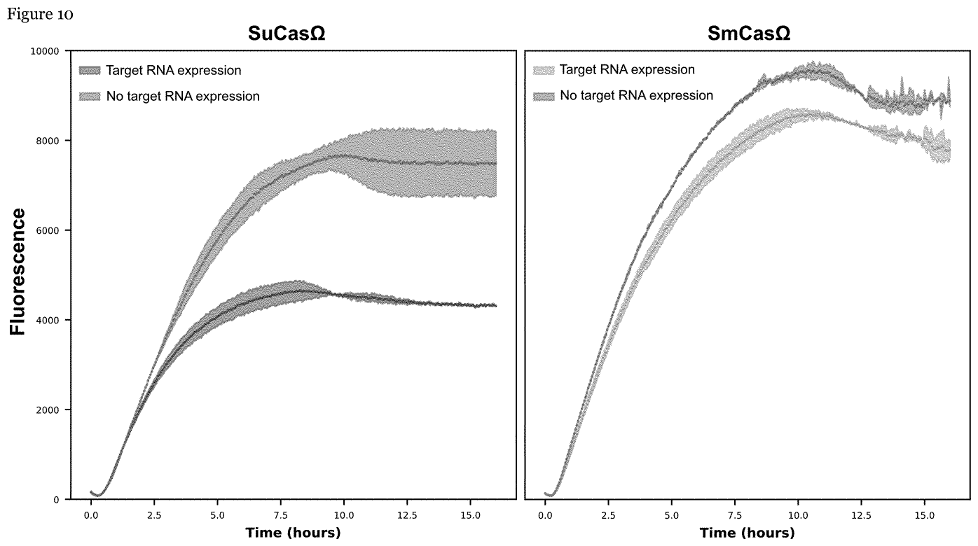

FIG. 10 shows that CasΩ nucleases exhibit RNA-triggered collateral activity in TXTL. RNA-triggered SuCasΩ and SmCasΩ activity was tested in cell-free transcription-translation (TXTL) reactions with a non-target plasmid DNA encoding a fluorescent GFP reporter. SuCasΩ and SmCasΩ nucleases and crRNA were expressed from plasmids. Target RNA was either expressed or not expressed from separate plasmids in the reactions. The results show that RNA recognition by CasΩ nucleases leads to a reduction in GFP fluorescence attributed to the collateral degradation of the non-target GFP-expressing reporter plasmid.

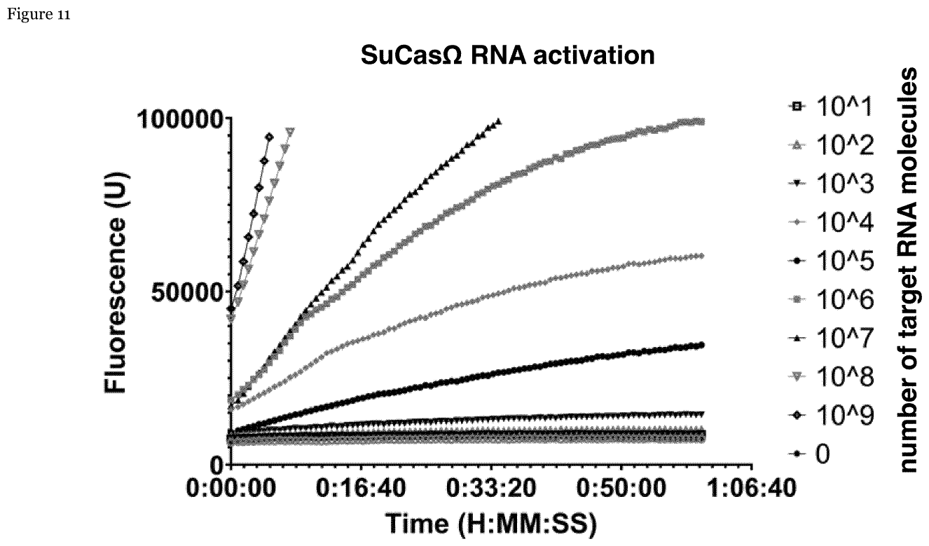

FIG. 11 shows that SuCasΩ is capable of detecting target RNA molecules. This property of CasΩ can be used to determine concentration of RNA as defined by crRNA in a test sample with unknown target RNA concentration.

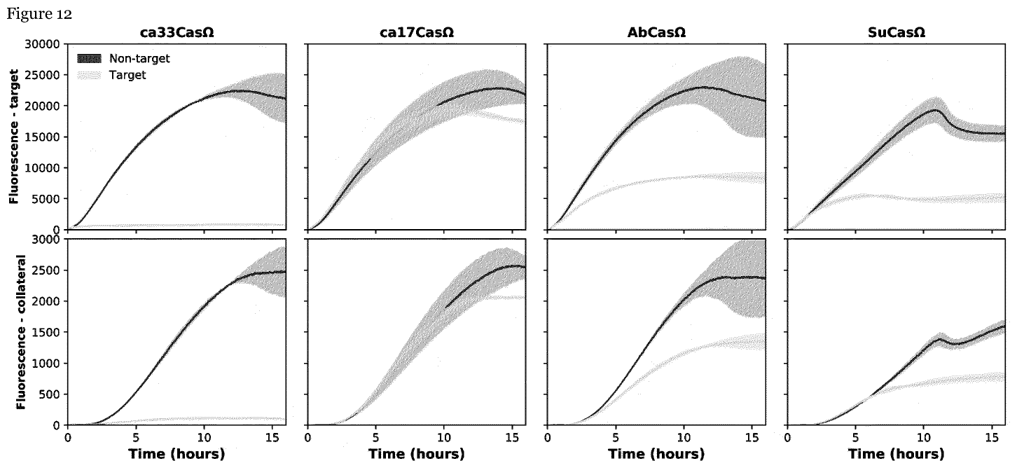

FIG. 12 shows that CasΩ nucleases from the SuCasΩ phylogenetic clade exhibit RNA-triggered on-target activity and collateral off-target activity in TXTL.

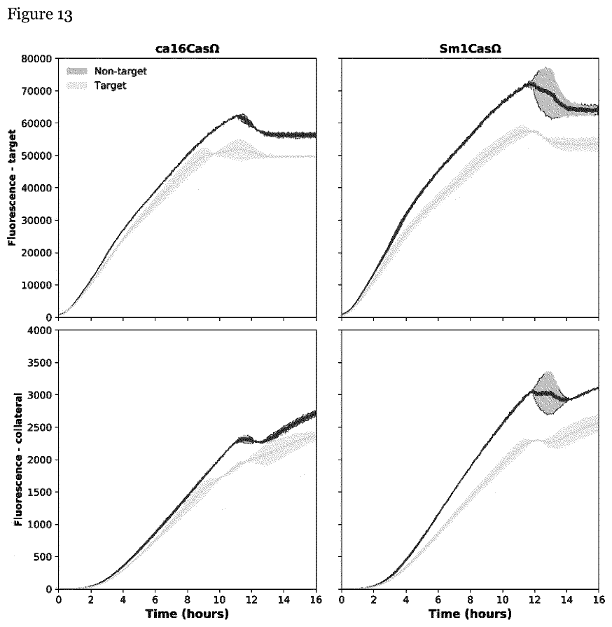

FIG. 13 shows that CasΩ nucleases from the SmCasΩ phylogenetic clade exhibit RNA-triggered on-target activity and collateral off-target activity in TXTL.

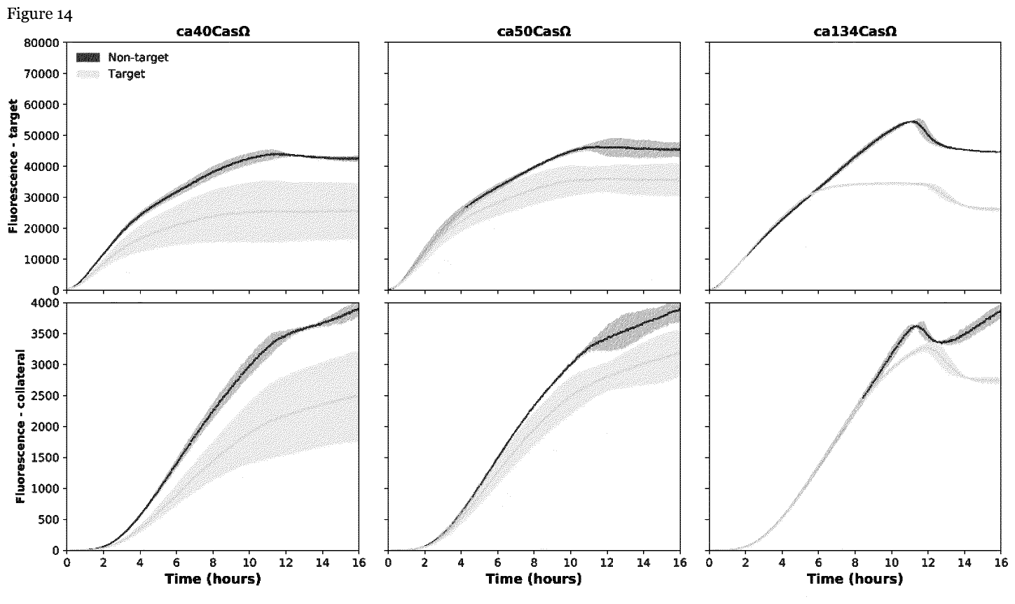

FIG. 14 shows that CasΩ nucleases from the ca40CasΩ phylogenetic clade exhibit RNA-triggered on-target activity and collateral off-target activity in TXTL.

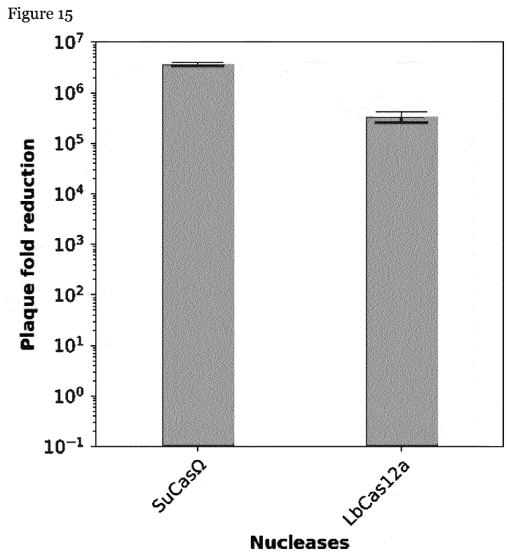

FIG. 15 shows that SuCasΩ nuclease reduced the number of T4 bacteriophage plaques in the presence of targeting crRNA compared to the non-targeting crRNA.

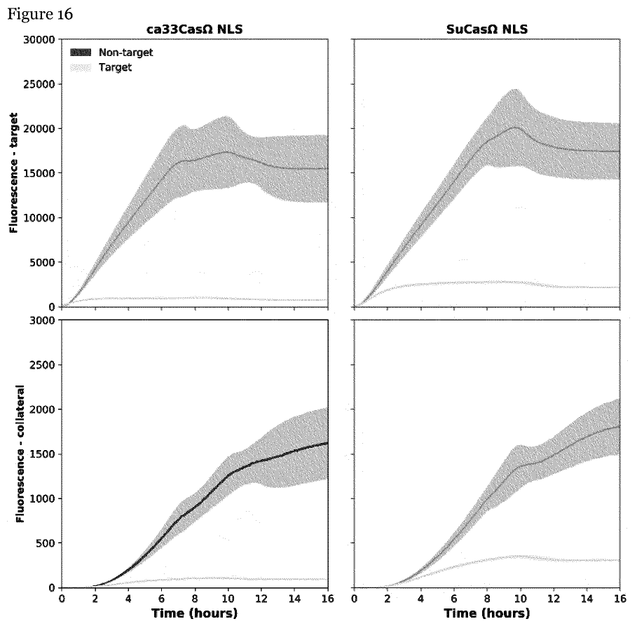

FIG. 16 shows that, CasΩ nucleases, such as ca33CasΩ and SuCasΩ containing Nuclear Localization Sequence (NLS) at the N-terminus and the C-terminus (N-NLS and C-NLS), exhibit RNA-triggered on-target activity and collateral off-target activity in TXTL.

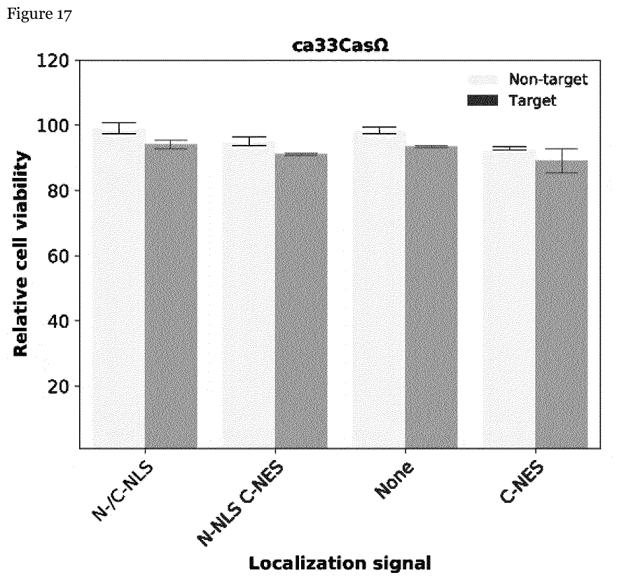

FIG. 17 shows that activity of ca33CasΩ reduced relative viability of the HEK293T cells.

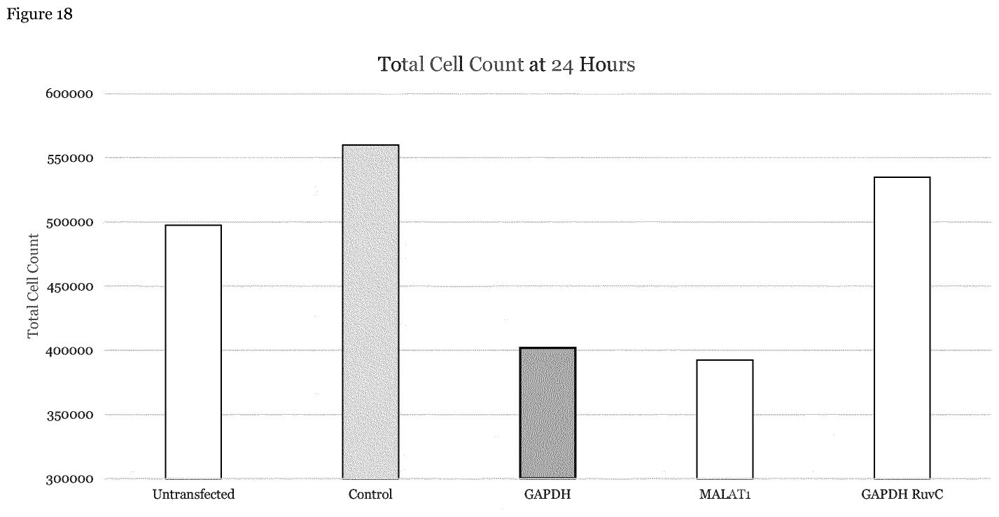

FIG. 18 shows hemocytometer data (see examples below). Untransfected Cells—HEK293 cells treated with lipofectamine but no DNA; Control—wild type (WT) SuCasΩ with a scrambled guide that does not target anything in mammalian cells; GAPDH—WT SuCasΩ with a guide that targets three separate regions on the GAPDH mRNA; MALAT1—WT SuCasΩ with a guide that targets three separate regions on the MALAT1 mRNA; and GAPDH RuvC—SuCasΩ E1070A mutant in the RuvC active site with a guide that targets three separate regions on the GAPDH mRNA.

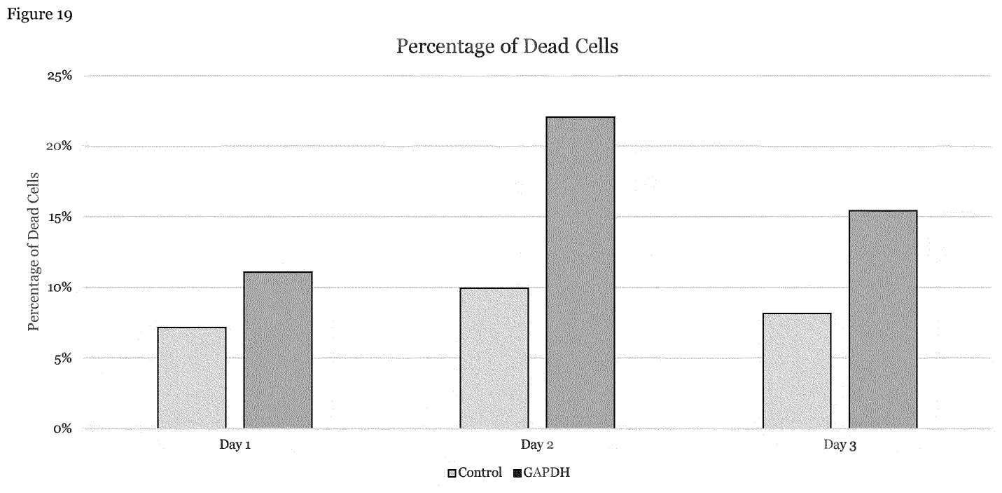

FIG. 19 shows hemocytometer data (see examples below). Control—WT SuCasΩ with a scrambled guide that does not target anything in mammalian cells; GAPDH—WT SuCasΩ with a guide that targets three separate regions on the GAPDH mRNA.

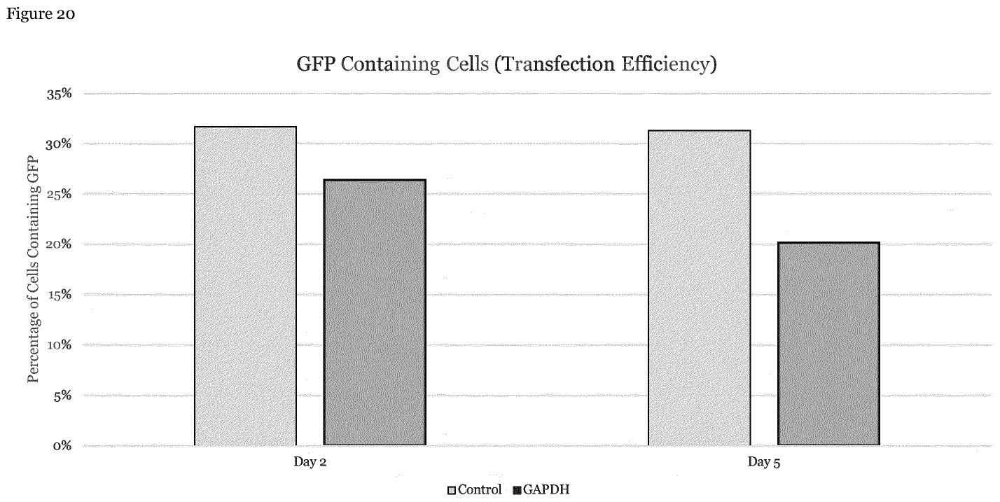

FIG. 20 shows flow cytometry data (see examples below). Control—WT SuCasΩ with a scrambled guide that does not target anything in mammalian cells; GAPDH—WT SuCasΩ with a guide that targets three separate regions on the GAPDH mRNA.

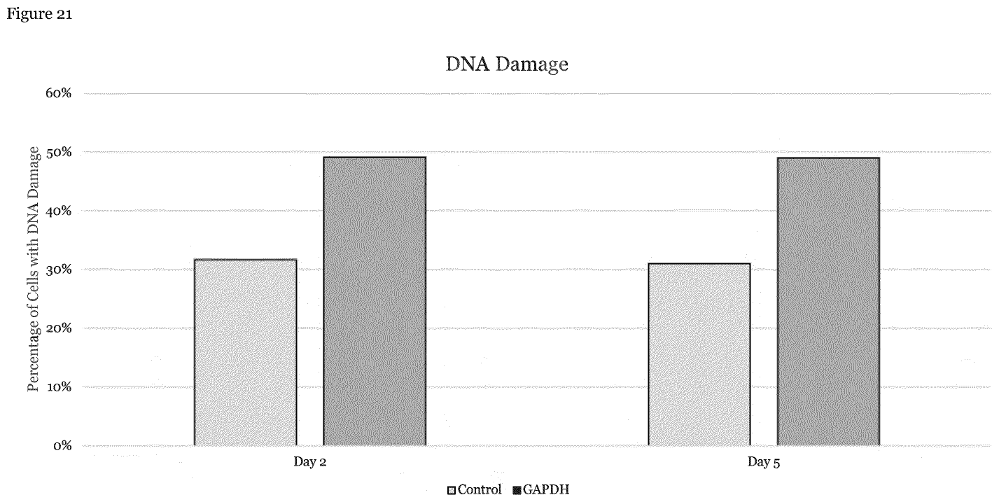

FIG. 21 shows flow cytometry data (see examples below). Control—WT SuCasΩ with a scrambled guide that does not target anything in mammalian cells; GAPDH—WT SuCasΩ with a guide that targets three separate regions on the GAPDH mRNA.

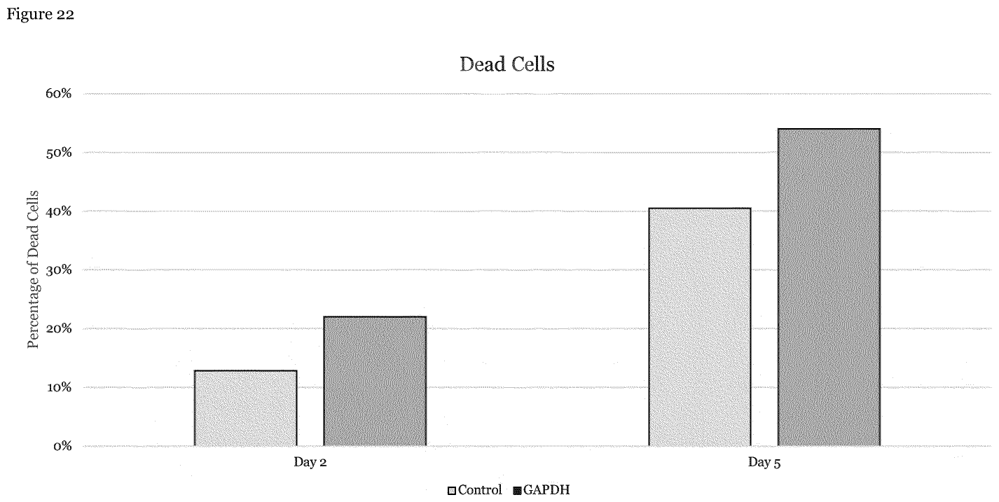

FIG. 22 shows cytometry data (see examples below). Control—WT SuCasΩ with a scrambled guide that does not target anything in mammalian cells; GAPDH—WT SuCasΩ with a guide that targets three separate regions on the GAPDH mRNA.

### EXAMPLES

### CasΩ Forms Three Distinct Clades Among Class 2 Type V CRISPR-Cas Nucleases

Maximum likelihood phylogeny of Class 2 Type V CRISPR-Cas protein sequences was generated, including three distinct monophyletic CasΩ clades, as typified by the representative nucleases SmCasΩ, SuCasΩ, and ca40CasΩ. CasΩ nucleases do not share the last common ancestor with Cas12a. Amino acid sequences of proteins were aligned using ClustalΩ. Phylogenetic reconstruction was generated using RAxML-NG using the following parameters:

--model JTT+G --bs-metric fbp,tbe --tree pars{60},rand{60}--seed 12345 --bs-trees autoMRE. TnpB amino acid sequenes served as an outgroup. See also FIG. 1.
Analysis of Amino Acid Conservation within CRISPR-SuCasΩ Nucleases

Nuclease orthologues from the SuCasΩ phylogenetic clade contain RuvC-I, RuvC-II, and RuvC-III catalytic motifs common to Type V CRISPR-Cas nucleases. SuCasΩ orthologues contain multiple conserved amino acid motifs absent from non-CasΩ nucleases, such as Cas12a. On average SuCasΩ orthologues share ≤10% sequence identity with Cas12a nucleases. Amino acids probabilities in each position within the alignment of 16 SuCasΩ orthologues are shown. Amino acid sequences of proteins were aligned using ClustalΩ. The amino acid logos and the corresponding probabilities were generated using WebLogo 3.

### Amino Acid Conservation Between the RuvC-I and RuvC-III Motifs in CRISPR-SuCasΩ Nucleases

Nuclease orthologues from the SuCasΩ phylogenetic clade exhibit unique amino acid composition, e.g., between the RuvC-I and RuvC-II catalytic motifs, comprising an insertion of multiple conserved amino acid motifs compared to non-CasΩ nucleases, such as Cas12a. Furthermore, SuCasΩ orthologues exhibit unique amino acid composition between the RuvC-II and RuvC-III catalytic motifs, comprising a deletion of amino acids compared to non-CasΩ nucleases, such as Cas12a. Relative entropy is shown in bits. High entropy indicates high certainty that a given amino acid is present in an orthologous motif based on the alignment of 16 SuCasΩ orthologues. Amino acid sequences of proteins were aligned using ClustalΩ. The amino acid logos and the corresponding entropy values were generated using WebLogo 3. See also FIG. 2.

Amino Acid Conservation within CRISPR-SmCasΩ Nucleases

Nuclease orthologues from the SmCasΩ phylogenetic clade contain RuvC-I, RuvC-II, and RuvC-III catalytic motifs common to Type V CRISPR-Cas nucleases. SmCasΩ orthologues contain multiple conserved amino acid motifs absent from non-CasΩ nucleases such as Cas12a. On average SmCasΩ orthologues share ≤10% sequence identity with Cas12a nucleases. Amino acids probabilities in each position within the alignment of 36 SmCasΩ orthologues are shown. Amino acid sequences of proteins were aligned using ClustalΩ. The amino acid logos and the corresponding probabilities were generated using WebLogo 3.

### Amino Acid Conservation Between the RuvC-I and RuvC-III Motifs in CRISPR-SmCasΩ Nucleases

Nuclease orthologues from the SmCasΩ phylogenetic clade exhibit unique amino acid composition, e.g., between the RuvC-I and RuvC-II catalytic motifs, comprising an insertion of multiple conserved amino acid motifs compared to non-CasΩ nucleases, such as Cas12a. Furthermore, SmCasΩ orthologues exhibit unique amino acid composition between the RuvC-II and RuvC-III catalytic motifs, comprising a deletion of amino acids compared to non-CasΩ nucleases, such as Cas12a. Relative entropy is shown in bits. High entropy indicates high certainty that a given amino acid is present in an orthologous motif based on the alignment of 36 SmCasΩ orthologues. Amino acid sequences were aligned using ClustalΩ. The amino acid logos and the corresponding entropy values were generated using WebLogo 3. See also FIG. 3.

Amino Acid Conservation within CRISPR-ca40CasΩ Nucleases

Nuclease orthologues from the ca40CasΩ phylogenetic clade contain RuvC-I, RuvC-II, and RuvC-III catalytic motifs common to Type V CRISPR-Cas nucleases. ca40CasΩ orthologues contain multiple conserved amino acid motifs absent from non-CasΩ nucleases such as Cas12a. On average ca40CasΩ orthologues share ≤10% sequence identity with Cas12a nucleases. Amino acids probabilities in each position within the alignment of 15 ca40CasΩ orthologues are shown. Amino acid sequences of proteins were aligned using ClustalΩ. The amino acid logos and the corresponding probabilities were generated using WebLogo 3.

### Amino Acid Conservation Between the RuvC-I and RuvC-III Motifs in CRISPR-ca40CasΩ Nucleases

Nuclease orthologues from the ca40CasΩ phylogenetic clade exhibit unique amino acid composition, e.g., between the RuvC-I and RuvC-II catalytic motifs, comprising an insertion of multiple conserved amino acid motifs compared to non-CasΩ nucleases, such as Cas12a. Furthermore, ca40CasΩ orthologues exhibit unique amino acid composition between the RuvC-II and RuvC-III catalytic motifs, comprising a deletion of amino acids compared to non-CasΩ nucleases, such as Cas12a. Relative entropy is shown in bits. High entropy indicates high certainty that a given amino acid is present in an orthologous motif based on the alignment of 15 ca40CasΩ orthologues. Amino acid sequences of proteins were aligned using ClustalΩ. The amino acid logos and the corresponding entropy values were generated using WebLogo 3. See also FIG. 4.

CasΩ Recognizes RNA and Cleaves RNA, ssDNA, and dsDNA In Vitro

As shown in FIG. 5, purified SuCasΩ and a designed guide RNA (crRNA) were combined with an unlabeled target or non-target RNA as well as labeled non-target single stranded DNA (ssDNA), double-stranded DNA (dsDNA), and single stranded RNA (ssRNA). (A) Only in the presence of the RNA target did SuCasΩ degrade the non-target ssDNA, dsDNA, and ssRNA. (B) In the presence of non-target RNA, SuCasΩ did not degrade the ssDNA, dsDNA, and ssRNA. This activity (in particular, RNA target recognition and dsDNA collateral degradation) represents an entirely unique activity for CRISPR nucleases.

For A) in FIG. 5, approximately 250 nM CasΩ:crRNA complex was mixed with 100 nM of either unlabeled targeted ssRNA or non-targeted ssRNA and 100 nM of labeled collateral substrate (non-target ssDNA, dsDNA, and ssRNA, *denotes 5′ FAM label.) Reactions were run in NEB 3.1 (50 mM Tris-HCl (pH 7.9), 100 mM NaCl, 10 mM MgCl2, 100 μg/mL BSA). Reactions were diluted to 10 μL with H₂O and incubated at 37° C. for 1 hour. Reactions were phenol:chloroform extracted before separation by 12% UREA-PAGE. The bands on the gel are of the labeled FAM substrates. For B) in FIG. 5, approximately 250 nM WT CasΩ:crRNA complex was mixed with 100 nM of either unlabeled target ssRNA or non-target ssRNA and 100 nM of 5′ FAM labeled non-target dsDNA. Reactions were run in NEB 3.1 (50 mM Tris-HCl (pH 7.9), 100 mM NaCl, 10 mM MgCl₂, 100 μg/mL BSA). Reactions were diluted to 10 μL with H₂O and incubated at 37° C. for 1 hour. Time points were taken at 1, 5, 10, 30, 60 min and were quenched by phenol:chloroform extraction before separation by 12% UREA-PAGE. The bands on the gel are of the labeled FAM substrates.

### RNA-Triggered DNA Degradation by CasΩ In Vitro Depends on the RuvC Domain

As shown in FIG. 6, SuCasΩ was mutated in two sites within the RuvC motifs associated with DNA cleavage. The cleavage assays were conducted as described in the previous figure. In this case, mutating the RuvC domain eliminated the RNA-triggered degradation of dsDNA. Approximately 250 nM E1064A/D1213A CasΩ:crRNA complex was mixed with 100 nM of either unlabeled target ssRNA or non-target ssRNA and 100 nM of 5′ FAM labeled non-target dsDNA. Reactions were run in NEB 3.1 (50 mM Tris-HCl (pH 7.9), 100 mM NaCl, 10 mM MgCl₂, 100 μg/mL BSA). Reactions were diluted to 10 μL with H₂O and incubated at 37° C. for 1 hour. Time points were taken at 1, 5, 10, 30, 60 min and were quenched by phenol:chloroform extraction before separation by 12% UREA-PAGE. The bands on the gel in FIG. 6 are of the labeled FAM substrates.

CasΩ Degrades ssDNA Following RNA Target Recognition In Vitro

RNA-triggered SuCasΩ activity was tested in vitro with non-target ssDNA. The ssDNA was labeled with a fluorophore for fluorescence detection. The results show that ssDNA is also degraded by triggered SuCasΩ. Target ssDNA and dsDNA did not trigger SuCasΩ activity. For the results as shown in FIG. 7, approximately 250 nM CasΩ:crRNA complex was mixed with 100 nM of labeled substrate (Target: ssRNA, ssDNA, or dsDNA). Reactions were run in NEB 3.1 (50 mM Tris-HCl (pH 7.9), 100 mM NaCl, 10 mM MgCl₂, 100 μg/mL BSA). Reactions were diluted to 10 μL with H₂O and incubated at 37° C. for 1 hour. Reactions were phenol: chloroform extracted and denatured in formaldehyde before separation by 12% UREA-PAGE in 0.5×MOPS buffer (10 mM MOPS, (pH 7.0), 2.5 mM Sodium Acetate, 0.5 mM EDTA). The bands on the gel are of the labeled FAM substrates.

### CasΩ Degrades Plasmid DNA Following RNA Target Recognition In Vitro

RNA-triggered SuCasΩ activity was tested in vitro with plasmid DNA. The plasmid was detected by running the nucleic acid products on an agarose gel and staining with ethidium bromide. The results show that plasmid DNA is also degraded by triggered SuCasΩ. For the results as shown in FIG. 8, approximately 100 nM CasΩ:crRNA complex was mixed with 100 nM of either unlabeled target ssRNA or non-target ssRNA and 40 nM of non-target plasmid (non-target pet27b TTTC). Reactions were run in NEB 3.1 (50 mM Tris-HCl (pH 7.9), 100 mM NaCl, 10 mM MgCl₂, 100 μg/mL BSA). Reactions were diluted to 10 μL with H₂O and incubated at 37° C. for 1 hour. Reactions were phenol:chloroform extracted before separation by electrophoresis in 1% agarose. Nucleic acids were visualized through staining with ethidium bromide.

CasΩ Impairs Growth Following Target Recognition in E. coli

The activity of SuCasΩ was assessed without selecting for the target plasmid or any plasmids. As shown in FIG. 9; (A-B) Transformation-fold reduction when transforming the SuCasΩ plasmid into cells already harboring the crRNA plasmid and a target/non-target plasmid. Different rPAMs and target mismatches were tested with or without selection for the target plasmid. SuCasΩ but not Cas12a reduced plasmid transformation even when not selecting for the target plasmid. (C) Assessing growth of E. coli cells expressing different nucleases under different selection conditions. SuCasΩ and LsCas13a but not LbCas12a reduced growth even in the absence of a selecting antibiotic. LsCas13a is known to collaterally degrade cellular RNAs upon target recognition, yielding a similar effect on growth. The impact of targeting by SuCasΩ compared to other nucleases was further assessed in E. coli. (D) Measuring the SOS response using the recA promoter driving GFP expression. GFP fluorescence was measured after 4-h induction of the nuclease and guide RNA, all in the absence of a selecting antibiotic. Only SuCasΩ significantly elicited the SOS response compared to a non-target control. (E) Assessing cell morphology and DNA content. Cells were stained with the DNA binding dye DAPI and assessed by flow cytometry analysis. Only cells with SuCasΩ targeting led to a bifurcation in the population, with some cells becoming filamentous while others becoming small and containing less DNA. Both reflect extensive DNA damage.

### CasΩ Nucleases Exhibit RNA-Triggered Collateral Activity in TXTL

RNA-triggered SuCasΩ and SmCasΩ activity was tested in cell-free transcription-translation (TXTL) reactions with a non-target plasmid DNA encoding a fluorescent GFP reporter. SuCasΩ and SmCasΩ nucleases and crRNA were expressed from plasmids. Target RNA was either expressed or not expressed from separate plasmids in the reactions. The results show that RNA recognition by CasΩ nucleases leads to a reduction in GFP fluorescence attributed to the collateral degradation of the non-target GFP-expressing reporter plasmid. See also FIG. 10, and FIGS. 12 to 14 and 16.

Example diagnostic use: CasΩ and a guide RNA designed to recognize SARS-CoV-2 are combined with a dsDNA probe fused to a fluorophore and quencher as well as RNA extracted from a patient sample. If the RNA sample contains the SARS-CoV-2 RNA, then the CasΩ:guide RNA complex will be triggered to degrade the dsDNA probe.

Release of the fluorophore from the quencher will give a fluorescent signal. This same approach could be used to distinguish SARS-CoV-2 variants.

Sequence-specific killing example: A chimeric antigen receptor is inserted into a patient T cell in the native receptor locus as part of immunotherapy, although editing only occurs in 1% of the cells. CasΩ-NLS and a guide RNA designed to recognize the unedited (but not the edited) locus is introduced through transient transfection of a plasmid or delivery of an RNP. Recognition of the RNA from the transcribed WT locus triggers extensive dsDNA degradation, killing off the unedited cells. The result is the population now comprises almost 100% edited cells. CasΩ could also be delivered to pathogens through conjugated plasmids or bacteriophages/phagemids, allowing sequence-specific killing within a microbial population or microbiome.

### CasΩ Nuclease is Capable of Detecting a Range of Target RNA Concentrations

FIG. 11 shows that SuCasΩ is capable of detecting target RNA molecules. Target RNA between 1×10⁰ and 1×10⁹ molecules was tested with 100 nM SuCasΩ-crRNA complex and 1 μM DNAse Alert (IDT, 11-02-01-04) in 1×NEB 3.1 buffer (NEB B7203). SuCasΩ-crRNA complex was formed by incubating SuCasΩ nuclease with crRNA at room temperature for 30 minutes. Detection was performed for 1 hour at room temperature by measuring fluorescence with excitation and emission wavelengths of 500/20 and 560/20, respectively. The results show that activation of SuCasΩ is dependent on the concentration of target RNA. This property of CasΩ can be utilized to determine concentration of RNA as defined by crRNA in a test sample with unknown target RNA concentration for diagnostic use.

CasΩ Nucleases from the SuCasΩ, SmCasΩ, and ca40CasΩ Phylogenetic Clades Exhibit RNA-Triggered On-Target Activity and Off-Target Collateral Activity in Cell-Free Transcription-Translation (TXTL) Assays

As shown in FIG. 12, CasΩ nucleases from the SuCasΩ phylogenetic clade exhibit RNA-triggered on-target activity and collateral off-target activity in TXTL (see also FIG. 10). RNA-triggered ca33CasΩ, ca17CasΩ, AbCasΩ, and SuCasΩ activity was tested with target plasmid DNA encoding GFP and non-target plasmid DNA encoding mCherry fluorescent reporters. ca33CasΩ, ca17CasΩ, AbCasΩ, and SuCasΩ nucleases and either targeting or non-targeting crRNA were expressed from plasmids. The results show that RNA recognition by CasΩ nucleases leads to a reduction in GFP fluorescence attributed to degradation of target GFP-expressing reporter plasmid and the cognate RNA and to a reduction in mCherry fluorescence attributed to collateral degradation of the non-target mCherry-expressing reporter plasmid and the cognate RNA.

As shown in FIG. 13, CasΩ nucleases from the SmCasΩ phylogenetic clade exhibit RNA-triggered on-target activity and collateral off-target activity in TXTL (see also FIG. 10). RNA-triggered ca16CasΩ and SmCasΩ activity was tested with target plasmid DNA encoding GFP and non-target plasmid DNA encoding mCherry fluorescent reporters. ca16CasΩ and SmCasΩ nucleases and either targeting or non-targeting crRNA were expressed from plasmids. The results show that RNA recognition by CasΩ nucleases leads to a reduction in GFP fluorescence attributed to degradation of target GFP-expressing reporter plasmid and the cognate RNA and to a reduction in mCherry fluorescence attributed to collateral degradation of the non-target mCherry-expressing reporter plasmid and the cognate RNA.

As shown in FIG. 14, CasΩ nucleases from the ca40CasΩ phylogenetic clade exhibit RNA-triggered on-target activity and collateral off-target activity in TXTL (see also FIG. 10). RNA-triggered ca40CasΩ, ca50CasΩ, and ca134CasΩ activity was tested with target plasmid DNA encoding GFP and non-target plasmid DNA encoding mCherry fluorescent reporters. ca40CasΩ, ca50CasΩ, and ca134CasΩ nucleases and either targeting or non-targeting crRNA were expressed from plasmids. The results show that RNA recognition by CasΩ nucleases leads to a reduction in GFP fluorescence attributed to degradation of target GFP-expressing reporter plasmid and the cognate RNA and to a reduction in mCherry fluorescence attributed to collateral degradation of the non-target mCherry-expressing reporter plasmid and the cognate RNA.

CasΩ Impairs T4 Phage Propagation Following Target Recognition in E. coli

The ability of SuCasΩ to inactivate bacterial viruses (bacteriophages) was evaluated in a plaque assay. As shown in FIG. 15, SuCasΩ nuclease reduced the number of T4 bacteriophage plaques in the presence of targeting crRNA compared to the non-targeting crRNA. E. coli bacteria expressed either SuCasΩ or LbCas12a nucleases and either crRNA targeting transcript of the e gene of the T4 bacteriophage or non-target crRNA. These bacteria were grown on agar plates and infected with the T4 bacteriophage. Plaques indicative of successful T4 bacteriophage infection and propagation were counted. Relative plaque reduction represents the ratio between the plaque counts obtained for the cultures expressing the nucleases with targeting crRNA and the cultures expressing the nucleases with non-targeting crRNA.

### CasΩ Nucleases Containing Nuclear Localization Signals Exhibit RNA-Triggered On-Target Activity and Off-Target Collateral Activity in TXTL

As shown in FIG. 16, CasΩ nucleases, such as ca33CasΩ and SuCasΩ containing Nuclear Localization Sequence (NLS) at the N-terminus and the C-terminus (N-NLS and C-NLS), exhibit RNA-triggered on-target activity and collateral off-target activity in TXTL (see also FIG. 10). Codons of the ca33CasΩ and SuCasΩ nuclease-encoding genes with NLS were optimized to reflect codon usage in mammalian cells. RNA-triggered activity of ca33CasΩ and SuCasΩ with C-NLS and N-NLS was tested with a target plasmid DNA encoding GFP and non-target plasmid DNA encoding mCherry fluorescent reporters. Targeting and non-targeting crRNA and ca33CasΩ and SuCasΩ nucleases with C-NLS and N-NLS were expressed from plasmids. The results show that RNA recognition by the CasΩ nucleases leads to a reduction in GFP fluorescence attributed to degradation of target GFP-expressing reporter plasmid and the cognate RNA and to a reduction in mCherry fluorescence attributed to collateral degradation of the non-target mCherry-expressing reporter plasmid and the cognate RNA.

### CasΩ Nuclease Reduces Cell Viability Following RNA Target Recognition in Mammalian Cells

The ability of RNA-triggered CasΩ to reduce cell-viability was tested in HEK293T cells. As shown in FIG. 17, activity of ca33CasΩ reduced relative viability of the HEK293T cells. The used ca33CasΩ nuclease-encoding gene was optimized to reflect codon usage in mammalian cells. The nuclease was either tagged with NLS at the N-terminus and the C-terminus (N-/C-NLS); NLS at the N-terminus and Nuclear Export Sequence (NES) and the C-terminus (N-NLS C-NES); neither NLS nor NES (None); or NES at the C-terminus (C-NES). Targeting and non-targeting crRNA and ca33CasΩ nuclease were expressed from plasmids. The target GFP RNA was expressed from a plasmid. Relative cell viability was measured as a percentage of the luminescent signal in the cells expressing ca33CasΩ nuclease and crRNA that targets GFP RNA compared to the cells expressing ca33CasΩ nuclease and non-targeting crRNA. CellTiter-Glo Luminescent Cell Viability Assay from Promega (G7570) was used. The ability of CasΩ to reduce viability of mammalian cells can be utilized for therapeutic use.

CasΩ in the Presence of Targeting crRNA and RNA Target Leads to Destruction of Mammalian Cells

For the data as shown in FIG. 18, 24 well plates were seeded with 5×10⁴ HEK293 cells which were allowed to adhere and grow for 48 hours in Eagle's minimal essential medium (MEM) with antibiotics. 500 ng of plasmid DNA encoding SuCasΩ nuclease and respective crRNA was combined with Lipofectamine 3000 (1.5 μl) and allowed to incubate for 15 minutes in 50 μl of opi-MEME with 1 μl p3000. The DNA-lipid complex was added to the cells. The cells were placed in the incubator at 37° C. and 5.0% CO₂. Following 24 hours, the media was removed and collected. The adherent cells were washed with PBS and 100 μl of trypsin was added. Cells were allowed to incubate at 37° C. and 5.0% CO₂ for five minutes. 400 μl of MEM was added to inactivate the trypsin. The cells, along with the collected media, were counted on a hemocytometer using Trypan blue. These results show that the presence of CasΩ and targeting guide leads to the destruction of mammalian cells, seen as the reduction in the total cell count in experimental conditions which contained an active immune system and guides designed to target specific sequences in the cell. There is a 20% and 30% reduction in total cell count in the GAPDH targeting condition and the MALAT1 targeting condition, respectively, compared to the controls.

For the data as shown in FIG. 19, 24 well plates were seeded with 5×10⁴ HEK293 cells which were allowed to adhere and grow for 48 hours in Eagle's minimal essential medium (MEM) with antibiotics. 500 ng of plasmid DNA encoding SuCasΩ nuclease and respective crRNA was combined with Lipofectamine 3000 (1.5 μl) and allowed to incubate for 15 minutes in 50 μl of opi-MEME with 1 μl p3000. The DNA-lipid complex was added to the cells. The cells were placed in the incubator at 37° C. and 5.0% CO₂. Following 24 hours, 48 hours, and 72 hours, the media was removed and collected. The adherent cells were washed with PBS and 100 μl of trypsin was added. Cells were allowed to incubate at 37° C. and 5.0% CO₂ for five minutes. 400 μl of MEM was added to inactivate the trypsin. The cells, along with the collected media, were counted on a hemocytometer using Trypan blue. On each day, the percentage of dead cells was found to be higher in the GAPDH targeting condition than the control condition. In reference to the control, there were between 50% to 120% more dead cells in the GAPDH targeting condition.

For the data as shown in FIG. 20, 6 well plates were seeded with 5×10⁵ HEK293 cells which were allowed to adhere and grow for 48 hours in Eagle's minimal essential medium (MEM) with antibiotics. 2.5 μg of plasmid DNA encoding SuCasΩ nuclease and respective crRNA was combined with Lipofectamine 3000 (7.5 μl) and allowed to incubate for 15 minutes in 250 μl of opi-MEME with 5 μl p3000. The DNA-lipid complex was added to the cells. The cells were placed in the incubator at 37° C. and 5.0% CO₂. Following 48 hours and 120 hours, the media was removed and collected. The adherent cells were washed with PBS and 500 μl of trypsin was added. Cells were allowed to incubate at 37° C. and 5.0% CO₂ for five minutes. 1.5 ml of MEM was added to inactivate the trypsin. The cells, along with the collected media, were counted on a hemocytometer using Trypan blue. 1×10⁶ cells were collected and added to a separate tube where they were spun down at 300×g for 3 minutes. The cells were washed once with 1 ml of PBS and spun down again. 1 μl of the reconstituted fluorescent reactive dye was added to the cell suspension and thoroughly mixed followed by a 30 minute long incubation on ice, protected from light. The cells were then washed with 1 ml of PBS and resuspended in 900 μl of PBS. They were then fixed with 2% formaldehyde for 60 minutes. Permeabilization was performed with 0.1% Triton X in 0.1% sodium citrate for 2 minutes. The cells were washed twice with PBS and resuspended in 50 μl TUNEL reaction mixture. The mixture was allowed to incubate for 60 minutes at 37° C. in a dark, humidified incubator. The samples were washed two additional times and resuspended in 500 μl PBS with 1% BSA. The cells were then run through a flow cytometer which monitored wavelengths for GFP, DAPI, and TUNEL. These data were taken from between 400 and 1400 events in each condition. The percentage of cells containing GFP stands as a rough estimation for transfection efficiency. Active CasΩ potentially results in the complete destruction of a cell, thereby resulting in a lower observed transfection efficiency when using the plasmid expressing the GAPDH targeting crRNA and SuCasΩ compared to the control.

For the data as shown in FIG. 21, 6 well plates were seeded with 5×10\hat{ }5 HEK293 cells which were allowed to adhere and grow for 48 hours in Eagle's minimal essential medium (MEM) with antibiotics. 2.5 μg of plasmid DNA encoding SuCasΩ nuclease and respective crRNA was combined with Lipofectamine 3000 (7.5 μl) and allowed to incubate for 15 minutes in 250 μl of opi-MEME with 5 μl p3000. The DNA-lipid complex was added to the cells. The cells were placed in the incubator at 37° C. and 5.0% CO₂. Following 48 hours and 120 hours, the media was removed and collected. The adherent cells were washed with PBS and 500 μl of trypsin was added. Cells were allowed to incubate at 37° C. and 5.0% CO₂ for five minutes. 1.5 ml of MEM was added to inactivate the trypsin. The cells, along with the collected media, were counted on a hemocytometer using Trypan blue. 1×10⁶ cells were collected and added to a separate tube where they were spun down at 300×g for 3 minutes. The cells were washed once with 1 ml of PBS and spun down again. 1 μl of the reconstituted fluorescent reactive dye was added to the cell suspension and thoroughly mixed followed by a 30 minute long incubation on ice, protected from light. The cells were then washed with 1 ml of PBS and resuspended in 900 μl of PBS. They were then fixed with 2% formaldehyde for 60 minutes. Permeabilization was performed with 0.1% Triton X in 0.1% sodium citrate for 2 minutes. The cells were washed twice with PBS and resuspended in 50 μl TUNEL reaction mixture. The mixture was allowed to incubate for 60 minutes at 37° C. in a dark, humidified incubator. The samples were washed two additional times and resuspended in 500 μl PBS with 1% BSA. The cells were then run through a flow cytometer which monitored wavelengths for GFP, DAPI, and TUNEL. Substantially more DNA damage was observed in the GAPDH targeting condition.

For the data as shown in FIG. 22, 6 well plates were seeded with 5×10⁵ HEK293 cells which were allowed to adhere and grow for 48 hours in Eagle's minimal essential medium (MEM) with antibiotics. 2.5 μg of plasmid DNA encoding SuCasΩ nuclease and respective crRNA was combined with Lipofectamine 3000 (7.5 μl) and allowed to incubate for 15 minutes in 250 μl of opi-MEME with 5 μl p3000. The DNA-lipid complex was added to the cells. The cells were placed in the incubator at 37° C. and 5.0% CO₂. Following 48 hours and 120 hours, the media was removed and collected. The adherent cells were washed with PBS and 500 μl of trypsin was added. Cells were allowed to incubate at 37° C. and 5.0% CO₂ for five minutes. 1.5 ml of MEM was added to inactivate the trypsin. The cells, along with the collected media, were counted on a hemocytometer using Trypan blue. 1×10⁶ cells were collected and added to a separate tube where they were spun down at 300×g for 3 minutes. The cells were washed once with 1 ml of PBS and spun down again. 1 μl of the reconstituted fluorescent reactive dye was added to the cell suspension and thoroughly mixed followed by a 30 minute long incubation on ice, protected from light. The cells were then washed with 1 ml of PBS and resuspended in 900 μl of PBS. They were then fixed with 2% formaldehyde for 60 minutes. Permeabilization was performed with 0.1% Triton X in 0.1% sodium citrate for 2 minutes. The cells were washed twice with PBS and resuspended in 50 μl TUNEL reaction mixture. The mixture was allowed to incubate for 60 minutes at 37° C. in a dark, humidified incubator. The samples were washed two additional times and resuspended in 500 μl PBS with 1% BSA. The cells were then run through a flow cytometer which monitored wavelengths for GFP, DAPI, and TUNEL. As expected, death rates increased from day 2 to day 5. Confluency and the lack of new media likely contributed to this general trend, however, death rates were higher in the GAPDH targeting condition, demonstrating that SuCasΩ is capable of causing programmable destruction of mammalian cells. This property of CasΩ can be adapted for therapeutic use.

1. A complex comprising a CasΩ nuclease and at least one preselected guide RNA designed for binding to at least one target RNA.

2. The complex according to claim 1, further bound to a target RNA molecule having a sequence that is at least 90% complementary to said guide RNA.

3. The complex according to claim 1, wherein said guide RNA comprises a sequence selected to be specific for a bacterium, a sequence selected to be specific for a virus, a sequence selected to be specific for a fungus, a sequence selected to be specific for a protozoan, a sequence selected to be specific for a genetic disorder, and/or a sequence selected to be specific for a proliferative disorder.

4. The complex according to claim 1, wherein said nuclease comprises a nuclear localization signal.

5. A method for cleaving a nucleic acid molecule selected from dsDNA, ssDNA, and RNA, comprising the steps of a) providing at least one CasΩ nuclease enzyme, b) providing at least one preselected guide RNA, c) forming a complex between the least one CasΩ nuclease enzyme and the at least one preselected guide RNA, d) binding of the complex of c) to a target RNA based on the at least one preselected guide RNA, and e) cleaving said nucleic acid molecule selected from dsDNA, ssDNA, and RNA by the at least one CasΩ nuclease enzyme.

6. A method for detecting at least one target RNA in a cell, tissue, cellular nucleus, and/or sample, said method comprising:
a) providing at least one ssDNA, dsDNA or RNA reporter nucleic acid in said cell, tissue, cellular nucleus, and/or sample,
b) contacting said cell, tissue, cellular nucleus, and/or sample with at least one complex according to claim 2, and
c) detecting a cleaving, cutting and/or nicking of said at least one ssDNA, dsDNA or RNA reporter nucleic acid, wherein detecting said cleaving the at least one reporter nucleic acid detects said at least one target RNA in said cell, tissue, cellular nucleus and/or sample.

7. The method according to claim 6, wherein detecting said cleaving, cutting and/or nicking of the at least one reporter nucleic acid comprises detecting a change in the signal of a suitable label, and/or detecting the said cleaved at least one reporter nucleic acid fragment itself.

8. The method according to claim 6 wherein the at least one target RNA is a mutated target RNA comprising at least one mutation compared to a control target RNA.

9. A method for modulating expression of at least one target RNA in a cell, tissue, cellular nucleus, and/or sample, wherein said at least one target RNA is selected from an mRNA, non-coding RNA and a viral RNA molecule, said method comprising:
a) contacting said cell, tissue, cellular nucleus, and/or sample with at least one complex according to claim 2, and
c) binding the complex of b) to the at least one target RNA and thereby altering the stability, processing, or translation of the at least one target RNA,
whereby the binding in c) modulates the expression of at least one target RNA in the cell, tissue, cellular nucleus, and/or sample.

10. A method for editing the sequence of at least one target RNA in a cell, tissue, cellular nucleus, and/or sample, wherein said at least one target RNA is selected from an mRNA, non-coding RNA and a viral RNA molecule, said method comprising:
a) contacting said cell, tissue, cellular nucleus, and/or sample with at least one complex between at least one modified and catalytically inactive CasΩ nuclease enzyme complexed with at least one RNA-modifying enzyme and at least one preselected guide RNA, wherein said at least one preselected guide RNA comprises a sequence that is at least 90% complementary to the at least one target RNA, and
c) binding the complex of b) to the at least one target RNA, and editing of the at least one target RNA by said at least one RNA-modifying enzyme.

11. The method according to claim 5, wherein the at least one target RNA comprises a nucleic acid sequence that is specific for a disease state, such as, for example, for cells selected from the group consisting of cells exhibiting a genetic disorder, cells exhibiting a proliferative disorder, such as cancer cells, immune cells that produce autoantibodies, cells infected with bacterial or viral pathogens, bacterial pathogens, protozoan pathogens, cells of microbiota, and contaminating bacteria or archaea.

12. (canceled)

13. A method for specifically inactivating an undesired cell or virus, comprising contacting said cell or virus with a complex according to claim 1, wherein said guide RNA is specifically selected for said undesired cell or virus to be inactivated.

14. A method for preventing and/or treating a disease comprising administering to a subject in need of such prevention and/or treatment an effective amount of the complex according to claim 3.

15. A method for cleaving a nucleic acid molecule selected from dsDNA, ssDNA, and RNA; for detecting at least one target RNA in a cell, tissue, cellular nucleus, and/or sample; for modulating expression of at least one target RNA in a cell, tissue, cellular nucleus, and/or sample; for editing the sequence of at least one target RNA in a cell, tissue, cellular nucleus, and/or sample; for specifically inactivating an undesired cell or virus, or for decontaminating a preparation from an undesired contaminant wherein said method comprises the use of a complex according to claim 1.

16. The complex according to claim 2, wherein said target RNA is flanked by at least one RNA protospacer-adjacent motif (rPAM).

17. The method according to claim 14, used to prevent and/or treat an infection, a genetic disorder, a proliferative disorder, or an autoimmune disease.
